(19) United States
(12) Patent Application Publication
(10) Pub. No.: US 2024/0058347 A1
(43) Pub. Date: Feb. 22, 2024

(54) DOSAGES FOR HDAC TREATMENT WITH REDUCED SIDE EFFECTS

(71) Applicant: VIRACTA THERAPEUTICS, INC., Cardiff, CA (US)

(72) Inventors: Robert MCRAE, Cardiff, CA (US); Gail L. BROWN, Cardiff, CA (US); David SLACK, Cardiff, CA (US); Marshelle Smith WARREN, Cardiff, CA (US); Richard TRAUGER, Cardiff, CA (US)

(21) Appl. No.: 17/766,643

(22) PCT Filed: Oct. 6, 2020
(86) PCT No.: PCT/US2020/054356

§ 371 Date: Apr. 5, 2022

Related U.S. Application Data
(60) Provisional application No. 62/911,862, filed on Oct. 7, 2019.

(51) Int. Cl. A61K 31/522; A61K 31/506; A61P 35/00

(52) U.S. Cl. CPC A61K 31/522; A61K 31/506; A61P 35/00

(57) ABSTRACT

Described herein are certain dosing schedules and amounts that effectively prevent and manage side effects associated with histone deacetylase inhibitor (HDACi) treatment. Optionally, these schedules and dosing regimens include treatment with an antiviral agent.

### CROSS-REFERENCE

This application claims the benefit of U.S. Provisional Application Ser. No. 62/911,862 filed on Oct. 7, 2019, which is incorporated by reference herein in its entirety.

### SUMMARY OF THE INVENTION

Described herein are certain dosing schedules and amounts that effectively prevent and manage side effects associated with histone deacetylase inhibitor (HDACi) treatment. Optionally, these schedules and dosing regimens include treatment with an antiviral agent.

The methods and uses described herein allow for dosing of HDACi at levels below a maximum tolerated or recommended dose, which allows for a significant reduction in side effects associated with HDACi treatment. Such side effects include nausea/vomiting, impairment of kidney function, and hematological side effects such as neutropenia, leukopenia, thrombocytopenia, and anemia. These dosage levels can be effectively combined with antiviral drugs to treat tumors or malignancies associated with a viral infection, or tumors or malignancies that comprise cancerous cells that comprise a latent viral infection.

Described herein, in one aspect, is a method of treating a cancer in an individual, the method comprising administering to the individual: (a) an effective amount of a histone deacetylase inhibitor (HDACi), wherein the HDACi is characterized by an elimination half-life of less than 30 hours; and (b) an effective amount of an antiviral drug; wherein the individual is treated according to a treatment schedule, wherein the individual is not administered the HDACi for at least one day of the treatment schedule. In certain embodiments, the HDACi is administered orally. In certain embodiments, the HDACi is selected from the list consisting of: vorinostat, romidepsin, mocetinostat, belinostat, pracinostat, givinostat, panobinostat, CUDC-101, CDX101, chidamide, and nanatinostat. In certain embodiments, the HDACi inhibits activity of a class I histone deacetylase. In certain embodiments, the HDACi is characterized by an elimination half-life of less than 24 hours. In certain embodiments, the HDACi is characterized by an elimination half-life of less than 12 hours. In certain embodiments, the HDACi is characterized by an elimination half-life of less than 12 hours. In certain embodiments, the HDACi is nanatinostat. In certain embodiments, the HDACi is administered at a total daily dose from about 10 milligrams to about 40 milligrams. In certain embodiments, the HDACi is administered at a total daily dose of about 10 milligrams. In certain embodiments, the HDACi is administered at a total daily dose of about 15 milligrams. In certain embodiments, the HDACi is administered at a total daily dose of about 20 milligrams. In certain embodiments, the HDACi is administered at a total daily dose of about 25 milligrams. In certain embodiments, the HDACi is administered at a total daily dose of about 30 milligrams. In certain embodiments, the HDACi is administered once per day. In certain embodiments, the cytotoxic activity of the antiviral agent is activated by a viral kinase. In certain embodiments, the viral kinase is a human herpes virus thymidine kinase, an Epstein-Barr virus thymidine kinase, an Epstein-Barr virus protein kinase, or a human cytomegalovirus protein kinase. In certain embodiments, the antiviral agent is selected from the list consisting of aciclovir, ganciclovir, valaciclovir, valganciclovir, and famciclovir. In certain embodiments, the antiviral agent is valganciclovir. In certain embodiments, the antiviral agent is administered at a total daily dose of 1,800 milligrams. In certain embodiments, the antiviral agent is administered at a total daily dose of 900 milligrams. In certain embodiments, the antiviral agent is administered at a total daily dose of 450 milligrams. In certain embodiments, the antiviral agent is administered every day of the treatment schedule. In certain embodiments, the antiviral agent is administered orally. In certain embodiments, the individual is not administered the HDACi for at least two days of the treatment schedule. In certain embodiments, the individual is not administered the HDACi for at least three days of the treatment schedule. In certain embodiments, the individual is not administered the HDACi for at least four days of the treatment schedule. In certain embodiments, the individual is not administered the HDACi for at least five days of the treatment schedule. In certain embodiments, the treatment schedule has a duration of one week. In certain embodiments, the treatment schedule is repeated. In certain embodiments, the HDACi is administered with food or a caloric substance. In certain embodiments, the cancer is a solid tissue cancer. In certain embodiments, the solid tissue cancer is salivary gland cancer, nasopharyngeal carcinoma, head and neck cancer, gastric cancer, a colorectal cancer, breast cancer, glioblastoma, prostate cancer, renal cancer, leiomyosarcoma, pancreatic cancer, or lung cancer. In certain embodiments, the solid tissue cancer is salivary gland cancer, nasopharyngeal carcinoma, head and neck cancer, gastric cancer, a colorectal cancer, or leiomyosarcoma. In certain embodiments, the cancer is a leukemia or a lymphoma. In certain embodiments, the leukemia or lymphoma is a B cell leukemia or lymphoma. In certain embodiments, the leukemia or lymphoma is a T cell leukemia or lymphoma. In certain embodiments, the leukemia or lymphoma is non-Hodgkin's lymphoma. In certain embodiments, the leukemia or lymphoma is Hodgkin's lymphoma. In certain embodiments, the leukemia or lymphoma is a cytomegalovirus virus positive leukemia or lymphoma. In certain embodiments, the leukemia or lymphoma is an Epstein-Barr virus positive leukemia or lymphoma. In certain embodiments, the individual is afflicted with thrombocytopenia. In certain embodiments, the individual has a platelet count of less than 50,000 platelets per microliter. In certain embodiments, the individual has an elevated creatinine level. In certain embodiments, the elevated creatinine level exceeds 1.1 mg/dL for a woman or 1.3 mg/dL for a man. In certain embodiments, the individual is selected for treatment according to the treatment schedule based on the presence of thrombocytopenia. In certain embodiments, the individual is selected based on a platelet count of less than 50,000 per microliter. In certain embodiments, the individual is selected for treatment according to the treatment schedule based on the presence of an elevated creatinine level. In certain embodiments, the elevated creatinine level exceeds 1.1 mg/dL for a woman and 1.3 mg/dL for a man.

In another aspect, described herein, is a method of treating an Epstein-Barr associated lymphoma in an individual, the method comprising administering to the individual: (a) an effective amount of nanatinostat; and (b) an effective amount of valganciclovir; wherein the individual is treated according to a treatment schedule, wherein the individual is not administered the nanatinostat for at least three days of the treatment schedule.

In another aspect, described herein, is a kit comprising: (a) an HDACi; and (b) an antiviral agent; wherein the kit comprises a plurality of oral dosage forms, the oral dosage forms comprising the HDACi and the antiviral agent co-formulated into a single oral dosage form, wherein at least one of the plurality of oral dosage forms comprises the antiviral agent and does not comprise the HDACi. In certain embodiments, the plurality of oral dosage forms are a pill, capsule, tablet, or gel cap. In certain embodiments, the HDACi is selected from the list consisting of: vorinostat, romidepsin, mocetinostat, belinostat, pracinostat, givinostat, panobinostat, CUDC-101, CDX101, chidamide, and nanatinostat. In certain embodiments, the HDACi inhibits activity of a class I histone deacetylase. In certain embodiments, the HDACi is characterized by an elimination half-life of less than about 24 hours. In certain embodiments, the HDACi is characterized by an elimination half-life of less than about 12 hours. In certain embodiments, the HDACi is characterized by an elimination half-life of less than about 8 hours. In certain embodiments, the HDACi is characterized by an elimination half-life of less than about 4 hours. In certain embodiments, the HDACi is characterized by an elimination half-life of less than about 2 hours. In certain embodiments, the HDACi is nanatinostat. In certain embodiments, the antiviral agent is activated by a viral kinase. In certain embodiments, the viral kinase is a human herpes virus thymidine kinase, an Epstein-Barr virus thymidine kinase, an Epstein-Barr virus protein kinase, or a human cytomegalovirus protein kinase. In certain embodiments, the antiviral agent is selected from the list consisting of aciclovir, ganciclovir, valaciclovir, valganciclovir, and famciclovir. In certain embodiments, the antiviral agent is valganciclovir. In certain embodiments, the plurality of oral dosage forms comprise about 900 mg of valganciclovir. In certain embodiments, the plurality of oral dosage forms comprise about 450 mg of valganciclovir. In certain embodiments, the plurality of oral dosage forms comprise about 20 mg of nanatinostat. In certain embodiments, the plurality of oral dosage forms comprise about 15 mg of nanatinostat. In certain embodiments, the plurality of oral dosage forms comprise about 10 mg of nanatinostat. In certain embodiments, the plurality comprise seven. In certain embodiments, one of the plurality of oral dosage forms comprises the antiviral agent and does not comprise the HDACi. In certain embodiments, two of the plurality of oral dosage forms comprises the antiviral agent and does not comprise the HDACi. In certain embodiments, three of the plurality of oral dosage forms comprises the antiviral agent and does not comprise the HDACi. In certain embodiments, four of the plurality of oral dosage forms comprises the antiviral agent and does not comprise the HDACi. In certain embodiments, five of the plurality of oral dosage forms comprises the antiviral agent and does not comprise the HDACi. In certain embodiments, the HDACi comprises nanatinostat and the antiviral agent comprises valganciclovir.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates rapid recovery of platelet counts in patients on nanatinostat dose hold.

FIG. 2 shows comparison of the potency of nanatinostat and entinostat in peripheral blood mononuclear cells of healthy donors. Percentage of cells with H3 acetylation, as determined by flow cytometry, is shown.

FIG. 3 shows time-course of H3 acetylation in PBMC from healthy donors analyzed as in FIG. 2.

FIG. 4 shows the cytotoxic activity of nanatinostat was increased by ˜40% when Nstat was combined with Ganciclovir. Additionally, this figure illustrates the duration of Nstat activity after 3 days of washout.

### DETAILED DESCRIPTION OF THE INVENTION

There is a need for methods of treating and/or preventing viral cancers and tumors. Many patients have latent infections in which a virus is present, but is not expressing viral proteins such as viral protein kinases, which activate common anti-viral drugs such as acyclovir, ganciclovir, and valganciclovir. A virus-inducing drug such as a histone deacetylase inhibitor (HDAC inhibitor—HDACi) can be used to re-induce the expression of a viral kinase in virus infected cells in the subject; the subject can then be treated with antiviral agents to eliminate latent viral infections. As herpesvirus and/or other latent viral infections can be associated with a variety of cancers and or tumors, eliminating the latent virus with HDACi and antiviral therapy is useful in preventing or treating such conditions.

While HDACi treatment holds promise for the treatment of cancers/tumors, HDACi treatment can be associated with dose limiting toxicities that negatively influence treatment decisions, patient compliance, and patient quality of life while being treated with HDACi. Some adverse effects associated with HDACi may even limit treatment with effective therapies that employ HDACi. Disclosed herein are methods of treating patients with an HDACi that reduces side-effects associated with HDACi treatment. These methods are particularly useful for treating patients with, or avoiding hematological side effects such as thrombocytopenia, neutropenia, Leukopenia, Anemia, or Lymphopenia. These methods are also useful for treating patients with, or avoiding side effects that indicate kidney toxicity, such as elevated creatinine.

Provided herein are methods and compositions for treating viral diseases and cancers and effectively reducing side-effects, thus improving quality of life and expanding treatment options. The cancer or tumor can be associated with latent viral infections. The methods can comprise the steps of administering an HDACi to the subject. The methods can comprise the steps of administering an HDACi and an antiviral agent to the subject. The method can comprise steps of administering a viral inducing agent, an antiviral agent, and one or more additional agents to a subject. The methods may include the co-administration of an oral HDAC inhibitor and an antiviral agent, either in the same or separate formulations.

The methods and compositions can be used to treat and/or prevent any of the cancers described herein. Any of the HDACi and/or antiviral agents described herein can be used in the methods and compositions of the provided invention. The HDAC inhibited can be any of a class I HDAC, for instance, HDAC1, HDAC2, and HDAC3. The HDAC inhibited can be a class IIb HDAC, for instance, HDAC10. The HDAC inhibitor can be a benzamide. The benzamide can be 4SC-202. The benzamide can be chidamide (also known as CS055 or HBI-8000). The HDAC inhibitor can be selected from the list consisting of: vorinostat, romidepsin, mocetinostat, belinostat, pracinostat, givinostat, panobinostat, CUDC-101, CDX101, chidamide, and nanatinostat. The HDAC inhibitor can be nanatinostat.

One or more additional agents described herein can be administered to a subject. An additional agent can be selected for administration based on the type of condition the subject has or is suspected of having.

Another aspect of the present invention relates to formulations, routes of administration and effective doses for pharmaceutical compositions comprising an agent or combination of agents, e.g., HDACi, antiviral agents, or, optionally, one or more additional agents. An HDACi, antiviral agent or, optionally, one or more additional agents can be administered to a subject in separate pharmaceutical compositions or can be co-formulated in a single pharmaceutical composition. The pharmaceutical combinations can be an oral formulation. The oral formulation can be a pill, capsule, or tablet. In certain embodiments, the pill, capsule or tablet can comprise a co-formulated dose of HDACi and antiviral agent. The anti-viral agent can be a herpes, Epstein-Barr virus, or cytomegalovirus antiviral agent. In certain embodiments, the antiviral agent is selected from the list consisting of aciclovir, ganciclovir, valaciclovir, valganciclovir, and famciclovir. In certain embodiments, the antiviral agent is valganciclovir.

Described herein, in one aspect, is a method of treating a cancer in an individual, the method comprising administering to the individual: (a) an effective amount of a histone deacetylase inhibitor (HDACi), wherein the HDACi is characterized by an elimination half-life of less than 30 hours; and (b) an effective amount of an antiviral drug; wherein the individual is treated according to a treatment schedule, wherein the individual is not administered the HDACi for at least one day of the treatment schedule.

In another aspect, described herein, is a method of treating an Epstein-Barr associated lymphoma in an individual, the method comprising administering to the individual: (a) an effective amount of nanatinostat; and (b) an effective amount of valganciclovir; wherein the individual is treated according to a treatment schedule, wherein the individual is not administered the nanatinostat for at least three days of the treatment schedule.

Also provided are methods relating to dosing schedules for administering an HDACi, an antiviral agent, or, optionally, one or more additional agents. One or more pharmaceutical compositions can be administered intermittently over a period of time. The schedule can encompass intermittent administration of an HDACi, and continuous administration of an antiviral agent. The intermittent administration of the HDACi can comprise an on and an off period, for example treating with the HDACi for 1, 2, 3, 4, or 5 days in a one-week period followed by not treating with the HDACi for 6, 5, 4, 3, or 2 days. Dosage for the on period can be dose orally once a day or twice a day. Dosages applied in this type of scheme can comprise 30, mg QD, 25 mg QD, 20 mg QD, 15 m QD, 10 mg QD, 5 mg BID, 10 mg BID, or 15 mg BID.

Also described herein are kits comprising oral dosage forms formulated to reflect the on and off period, with continuous administration of the antiviral. For example, packaging with a weeks-worth or more of treatment, wherein the “on days” are oral dosage forms combining an HDACi and an antiviral, and the “off” days are oral dosage forms that comprise only the antiviral. These types of kits and packaging can increase convenience and thus compliance for patients.

In another aspect, described herein, is a kit comprising: (a) an HDACi; and (b) an antiviral agent; wherein the kit comprises a plurality of oral dosage forms, the oral dosage forms comprising the HDACi and the antiviral agent co-formulated into a single oral dosage form, wherein at least one of the plurality of oral dosage forms comprises the antiviral agent and does not comprise the HDACi.

### Definitions

The terms “viral,” “virus-associated,” and “virally-induced” with reference to disorders are used interchangeably throughout the instant specification.

The term “obtaining” as in “obtaining the composition” is intended to include purchasing, synthesizing, or otherwise acquiring the composition (or agent(s) of the composition).

The terms “comprises”, “comprising”, are intended to have the broad meaning ascribed to them and can mean “includes”, “including” and the like.

The term “subject”, “patient” or “individual” are used interchangeably herein and refer to mammals and non-mammals, e.g., suffering from a disorder described herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods and compositions provided herein, the mammal is a human.

The terms “treat,” “treating” or “treatment,” and other grammatical equivalents as used herein, include alleviating, inhibiting or reducing symptoms, reducing or inhibiting severity of, reducing incidence of, prophylactic treatment of, reducing or inhibiting recurrence of, delaying onset of, delaying recurrence of, abating or ameliorating a disease or condition symptoms, ameliorating the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. The terms further include achieving a therapeutic benefit. By therapeutic benefit is meant eradication or amelioration of the underlying disorder being treated, and/or the eradication or amelioration of one or more of the physiological symptoms associated with the underlying disorder such that an improvement is observed in the patient.

Dosages are referred to herein as QD or BID. QD refers to dosing once a day. BID refers to dosing twice daily of the listed dose. For example, 10 mg BID refers to two 10 mg dosage units delivered daily. BID doses may be spaced apart such that they are at least about 16, 12, 10, 8, or 4 hours apart.

The terms “prevent,” “preventing” or “prevention,” and other grammatical equivalents as used herein, include preventing additional symptoms, preventing the underlying metabolic causes of symptoms, inhibiting the disease or condition, e.g., arresting the development of the disease or condition and are intended to include prophylaxis. The terms further include achieving a prophylactic benefit. For prophylactic benefit, the compositions are optionally administered to a patient at risk of developing a particular disease, to a patient reporting one or more of the physiological symptoms of a disease, or to a patient at risk of reoccurrence of the disease.

The terms “effective amount” or “therapeutically effective amount” as used herein, refer to a sufficient amount of at least one agent being administered which achieve a desired result, e.g., to relieve to some extent one or more symptoms of a disease or condition being treated. In certain instances, the result is a reduction and/or alleviation of the signs, symptoms, or causes of a disease, or any other desired alteration of a biological system. In certain instances, an “effective amount” for therapeutic uses is the amount of the composition comprising an agent as set forth herein required to provide a clinically significant decrease in a disease. An appropriate “effective” amount in any individual case is determined using any suitable technique, such as a dose escalation study.

The terms “administer,” “administering”, “administration,” and the like, as used herein, refer to the methods that are used to enable delivery of agents or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Administration techniques that in some instances are employed with the agents and methods described herein include, e.g., as discussed in Goodman and Gilman, The Pharmacological Basis of Therapeutics (current edition), Pergamon; and Remington's, Pharmaceutical Sciences (current edition), Mack Publishing Co., Easton, Pa. In certain embodiments, the agents and compositions described herein are administered orally. In some embodiments, the compositions described herein are administered parenterally.

The term “pharmaceutically acceptable” as used herein, refers to a material that does not abrogate the biological activity or properties of the agents described herein, and is relatively nontoxic (i.e., the toxicity of the material significantly outweighs the benefit of the material). In some instances, a pharmaceutically acceptable material is administered to an individual without causing significant undesirable biological effects or significantly interacting in a deleterious manner with any of the components of the composition in which it is contained.

The term “pharmaceutically acceptable excipient,” as used herein, refers to carriers and vehicles that are compatible with the active ingredient (for example, a compound of the invention) of a pharmaceutical composition of the invention (and preferably capable of stabilizing it) and not deleterious to the subject to be treated. For example, solubilizing agents that form specific, more soluble complexes with the compounds of the invention can be utilized as pharmaceutical excipients for delivery of the compounds. Suitable carriers and vehicles are known to those of extraordinary skill in the art. The term “excipient” as used herein will encompass all such carriers, adjuvants, diluents, solvents, or other inactive additives. Suitable pharmaceutically acceptable excipients include, but are not limited to, water, salt solutions, alcohol, vegetable oils, polyethylene glycols, gelatin, lactose, amylose, magnesium stearate, talc, silicic acid, viscous paraffin, perfume oil, fatty acid monoglycerides and diglycerides, petroethral fatty acid esters, hydroxymethyl-cellulose, polyvinylpyrrolidone, etc. The pharmaceutical compositions of the invention can also be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like, which do not deleteriously react with the active compounds of the invention.

The invention can be understood more fully by reference to the following detailed description and illustrative examples, which are intended to exemplify non-limiting embodiments of the invention.

In certain instances, the methods described herein and reduced maximum tolerated doses are for use in treating viral conditions in combination with one or more antiviral drugs. In certain embodiments, the viral condition is a herpesvirus condition. In certain embodiments, the viral condition is a cancer.

### Herpesviruses

Herpesviruses are a large family of DNA viruses that include Herpes simplex viruses (HSVs) 1 and 2, Varicella zoster virus, Epstein-Barr virus (EBV), cytomegalovirus (CMV) and human herpesviruses (HHVs) 6A, 6B, 7 and 8, which can cause various diseases in humans. Herpesviruses have two stages of replication, the lytic and the latent. Soon after primary infection, immunological surveillance by the host force herpesviruses to enter the latent state of infection, where only a few selected genes are expressed. Conventional anti-herpesvirus drugs, such as ganciclovir, acyclovir, etc., fail to act on these latently-infected cells because the viral enzyme thymidine kinase (TK) or protein kinase (PK), which is necessary for the conversion of the prodrugs to their toxic metabolites, is not expressed in latently-infected cells. Provided herein, in some embodiments, is a combination treatment wherein lytic replication is induced and antiviral agents are administered concurrently.

For example, previous studies using patient-derived cells in vitro, and also from phase I/II clinical studies on a series of patients with EBV-associated lymphomas, have clearly shown the great promise of this combination therapy approach. Strong epidemiological association of Epstein—Barr virus (EBV) with various human lymphoid malignancies and in vitro studies demonstrating tumorigenic activity of many EBV latent gene products suggest a causal relationship between EBV and these diseases. However, as EBV maintains a latent state of infection in these lymphomas, typical anti-herpesviral drugs, such as the nucleoside analogs ganciclovir (GCV) or acyclovir, are ineffective as these pro-drugs require expression of a lytic phase EBV protein, thymidine kinase (TK) or protein kinase (EBV-PK), for their activity. Therefore, selective induction of EBV lytic-phase gene expression in lymphoma cells that harbor latent EBV, coupled with simultaneous exposure to antiviral agents, has been advanced as promising targeted therapy, because of resulting targeting of cytotoxicity to the EBV-infected tumor cells.

A variety of agents including short-chain fatty acids and chemotherapeutic drugs, have been used to induce EBV lytic-phase infection in cultured cells, but these in vitro studies have generally not resulted in clinical application. For instance, arginine butyrate and GCV has been used to treat EBV-positive lymphoid malignancies in a recent Phase I/II clinical trial. In this study of 15 patients with relapsed or refractory EBV-positive lymphoid tumors, 4 patients achieved complete tumor remissions and 6 patients' partial tumor remissions. However, the rapid metabolism of butyrate requires continuous IV administration of high doses. Butyrate has pan-HDAC inhibitory activity, and it has been established that this activity is responsible for the induction of the EBV-TK protein. HDAC inhibitors may induce both EBV-TK and EBV-PK in EBV infected tumors. HDAC inhibitors may increase the activity of the CMV promoter in tumor cells. HDAC inhibitors may increase transcription of latent Herpes simplex virus genes in cell culture and tumors. In recent years, several potent HDAC inhibitors (HDACi) have been tested in the clinic as anti-cancer agents. In some instances, HDAC inhibitors induce lytic phase gene expression in viruses and kill virus-infected cells in combination with antiviral agents. In certain instances, HDAC inhibitors, including some new, highly-potent compounds, induce EBV lytic phase gene expression and kill EBV-infected cells in combination with antiviral agents. In some instances, HDAC inhibitors induce lytic phase gene expression in herpesviruses and kill virus-infected cells in combination with antiviral agents.

In certain embodiments, the methods described herein are for use in treating a cancer associated with one or more Herpesviruses including but not limited to Herpes simplex viruses (HSVs) 1 and 2, Varicella zoster virus, Epstein-Barr virus (EBV), cytomegalovirus (CMV) and human herpesviruses (HHVs) 6A, 6B, 7 and 8. In certain embodiments, the methods described herein are for use in treating a cancer in an individual latently infected with one or more Herpesviruses including but not limited to Herpes simplex viruses (HSVs) 1 and 2, Varicella zoster virus, Epstein-Barr virus (EBV), cytomegalovirus (CMV) and human herpesviruses (HHVs) 6A, 6B, 7 and 8.

### Histone Deacetylase Inhibitors

The methods of the provided invention comprise use of one or more pharmaceutical compositions provided herein comprising a histone deacetylase inhibitor (HDACi) to induce expression of a gene product in a virus-infected cell. The gene product expressed can be a viral enzyme or a cellular enzyme or activity that is largely expressed in virus-infected cells. Expression products that can be targeted include viral kinases, including protein kinases and the like, enzymes involved with DNA replication, for example, for repair or replication of the genome, assembly of complete virus particles, generation of viral membrane or walls, RNA transcription or protein translation or combinations of these activities. Interference with these processes can be performed by inducing and then acting on a viral gene product and, preferably, a critical viral gene product in the process.

In certain embodiments, the HDACi that can be used in conjunctions with the dosing schedules described herein is an HDACi with a short elimination half-life. In certain embodiments, the elimination half-life is less than 36 hours. In certain embodiments, the elimination half-life is less than 30 hours. In certain embodiments, the elimination half-life is less than 24 hours. In certain embodiments, the elimination half-life is less than 16 hours. In certain embodiments, the elimination half-life is less than 14 hours. In certain embodiments, the elimination half-life is less than 12 hours. In certain embodiments, the elimination half-life is less than 11 hours. In certain embodiments, the elimination half-life is less than 10 hours. In certain embodiments, the elimination half-life is less than 9 hours. In certain embodiments, the elimination half-life is less than 8 hours. In certain embodiments, the elimination half-life is less than 7 hours. In certain embodiments, the elimination half-life is less than 6 hours. In certain embodiments, the elimination half-life is less than 5 hours. In certain embodiments, the elimination half-life is less than 4 hours. In certain embodiments, the elimination half-life is between 2 and 16 hours, 2 and 14 hours, 2 and 12 hours, 2 and 11 hours, 2 and 10 hours, 2 and 9 hours, 2 and 8 hours, 2 and 7 hours, 2 and 6 hours, 2 and 5 hours, or 2 and 4 hours. In certain embodiments, the elimination half-life is between 3 and 16 hours, 3 and 14 hours, 3 and 12 hours, 3 and 11 hours, 3 and 10 hours, 3 and 9 hours, 3 and 8 hours, 3 and 7 hours, 3 and 6 hours, 3 and 5 hours, or 3 and 4 hours. In certain embodiments, the elimination half-life is between 4 and 16 hours, 4 and 14 hours, 4 and 12 hours, 4 and 11 hours, 4 and 10 hours, 4 and 9 hours, 4 and 8 hours, 4 and 7 hours, 4 and 6 hours, or 4 and 5 hours.

In some embodiments, the viral inducing agent is an HDAC inhibitor. In certain embodiments, the HDAC inhibitor is selected from the list consisting of: vorinostat, romidepsin, mocetinostat, belinostat, pracinostat, givinostat, panobinostat, CUDC-101, CDX101, chidamide, and nanatinostat. In certain embodiments, the HDAC inhibitor is vorinostat. In certain embodiments, the HDAC inhibitor is romidepsin. In certain embodiments, the HDAC inhibitor is mocetinostat. In certain embodiments, the HDAC inhibitor is belinostat. In certain embodiments, the HDAC inhibitor is pracinostat. In certain embodiments, the HDAC inhibitor is givinostat. In certain embodiments, the HDAC inhibitor is panobinostat. In certain embodiments, the HDAC inhibitor is CUDC-101. In certain embodiments, the HDAC inhibitor is CDX101. In certain embodiments, the HDAC inhibitor is chidamide. In certain embodiments, the HDAC inhibitor is nanatinostat. Nanatinostat is also referred to as CHR-3996 and VRx-3996, which is chemically identical). The chemical formula of nanatinostat is (2-(6-{[(6-Fluoroquinolin-2-yl)methyl]amino}-3-azabicyclo[3.1.0]hex-3-yl)-N-hydroxypyrimidine-5-carboxamide). Nanatinostat is a selective Class I HDAC inhibitor and is disclosed in U.S. Pat. No. 7,932,246, which is incorporated by reference herein in its entirety.

In some embodiments, the viral inducing agent is an HDACi. In certain embodiments, the HDACi prevents Histone 3 deacetylation in the peripheral blood mononuclear cells of the individual to which it is administered. This prevention of deacetylation is reflected by higher steady-state levels of histone acetylation.

### Induced Genes Including Viral-Associated Genes

HDACi (agents that induce expression) may act directly on the viral genome or indirectly through a cellular factor required for viral expression. For example, viral gene expression can be regulated through the regulation of the expression of viral transcription factors such as ZTA, RTA, tat, and tax, cellular transcription factors such as AP-1, AP-2, Sp1, NF-κB, and other transcriptional activators and/or repressors (factors), co-activators and co-repressors, histone acetylators and deacetylators, DNA methylases and demethylases, oncogenes or proto-oncogenes, or protein kinase C. These proteins act to regulate and thereby control expression of specific viral and/or other cellular genetic elements. According to the methods of the invention, control over their expression can lead to control over the infection. Other gene products, both viral and cellular in origin, whose expression can be regulated with inducing agents include proteases, polymerases, reverse transcriptases, cell-surface receptors, major histocompatibility antigens, growth factors, and combination of these products.

Additional genes whose expression or transcriptional regulation are altered in the presence of butyric acid include the oncogenes myc, ras, myb, abl and src. The activities of these gene products, as well as the activities of other oncogenes, are described in Slamon, J. D., et al. 1984 Science 224:256-62. Anti-proliferative activity also includes the ability to repress tumor angiogenesis through the blockade of angiogenesis factor activity, production or release, transcriptional regulation, or the ability to modulate transcription of genes under angiogenesis or growth factor or hormonal control. Either would be an effective therapy, particularly against both prostatic neoplasia and breast carcinomas. Further activities that effect transcription and/or cellular differentiation include increased intracellular cAMP levels, inhibition of histone acetylation, and inhibition of genomic methylation. Each of these activities is directly related to gene expression, and increased expression can sensitize infected cells to a specific anti-viral agent.

In other embodiments, inducing agents include HDAC inhibitors that induce EBV-PK activity (also known BGLF4) in EBV infected tumors. Expression of EBV-PK/BGLF4 sensitizes a cell to an antiviral agent. In certain instances, HDAC inhibitors induce EBV-PK. In some instances, HDAC inhibitors induce EBV-TK and/or EBV-PK. In some instances, HDAC inhibitors induce HSV-TK and/or HSV-PK. In some instances, HDAC inhibitors induce CMV-PK.

Preliminary in vitro studies according to the invention demonstrate that induction of EBV-TK activity in EBV-immortalized B-cells and patient-derived tumor cells using these drugs is possible, and that these previously resistant cells are rendered susceptible to ganciclovir therapy. Treatment of patients with viral-associated tumors such as EBV with inducing agents to induce the expression of EBV-TK/EBV-PK, and GCV, to eliminate EBV-TK/EBV-PK expressing tumor cells, is an effective, non-toxic therapy. This therapeutic regimen does not depend on the associated viral genome being the cause of the tumor. Without wishing to be bound by theory, it is believed that just the presence of the EBV genome in latent form would make the tumor susceptible to this combination protocol.

In some embodiments, an inducing agent induces viral gene expression by more than 4-fold after 24 h of treatment. In certain embodiments, an HDAC inhibitor induces TK or EBV-PK expression by more than 4-fold after 24 h of treatment. In some embodiments, an HDAC inhibitor induces viral gene expression after about 48 h, about 36 h, about 24 h, about 18 h, about 12 h, about 8 h, about 6 h, about 4 h, about 3 h, about 2 h, about 1 h, or about 30 minutes. In certain embodiments, an HDAC inhibitor induces viral gene expression in less than 48 h, less than 36 h, less than 24 h, less than 18 h, less than 12 h, less than 8 h, less than 6 h, less than 4 h, less than 3 h, less than 2 h, less than 1 h, or less than 30 minutes. In some embodiments, an HDAC inhibitor induces viral gene expression in more than 48 h, more than 36 h, more than 24 h, more than 18 h, more than 12 h, more than 8 h, more than 6 h, more than 4 h, more than 3 h, more than 2 h, more than 1 h, or more than 30 minutes. In certain embodiments, an HDAC inhibitor induces viral gene expression after more than 30 minutes and less than 24 h.

### Antiviral Agents

Anti-viral agents that can be used in the compositions and methods of the provided invention can include, for example, substrates and substrate analogs, inhibitors and other agents that severely impair, debilitate or otherwise destroy virus-infected cells. Substrate analogs include amino acid and nucleoside analogs. Substrates can be conjugated with toxins or other viricidal substances. Inhibitors include integrase inhibitors, protease inhibitors, polymerase inhibitors and transcriptase inhibitors such as reverse transcriptase inhibitors.

Antiviral agents that can be used in the compositions and methods of the provided invention can include, for example, ganciclovir, valganciclovir, oseltamivir (Tamiflu™) zanamivir (Relenza™), abacavir, aciclovir, acyclovir, adefovir, amantadine, amprenavir, ampligen, arbidol, atazanavir, atripla, boceprevir, cidofovir, combivir, darunavir, delavirdine, didanosine, docosanol, edoxudine, efavirenz, emtricitabine, enfuvirtide, entecavir, famciclovir, fomivirsen, fosamprenavir, foscarnet, fosfonet, fusion inhibitors (e.g., enfuvirtide), ibacitabine, imunovir, idoxuridine, imiquimod, indinavir, inosine, integrase inhibitor, interferon type III, interferon type II, interferon type I, interferon, lamivudine, lopinavir, loviride, maraviroc, moroxydine, nelfinavir, nevirapine, nexavir, nucleoside analogues, peginterferon alfa-2a, penciclovir, peramivir, pleconaril, podophyllotoxin, protease inhibitor, raltegravir, reverse transcriptase inhibitor, ribavirin, rimantadine, ritonavir, pyrimidine antiviral, saquinavir, stavudine, synergistic enhancer (antiretroviral), tenofovir, tenofovir disoproxil, tipranavir, trifluridine, trizivir, tromantadine, truvada, valaciclovir (Valtrex™), vicriviroc, vidarabine, viramidine, zalcitabine, and zidovudine.

In a specific embodiment, the antiviral agent is acyclovir, ganciclovir, or valganciclovir.

In some embodiments, the antiviral agent is a nucleoside analog. Examples of nucleoside analogs include acyclovir (ACV), ganciclovir (GCV), valganciclovir, famciclovir, foscarnet, ribavirin, zalcitabine (ddC), zidovudine (AZT), stavudine (D4T), larnivudine (3TC), didanosine (ddI), cytarabine, dideoxyadenosine, edoxudine, floxuridine, idozuridine, inosine pranobex, 2′-deoxy-5-(methylamino)uridine, trifluridine and vidarabine. Examples of a few protease inhibitors that show particular promise in human therapy include saquinivir, ritonavir and indinavir. Other anti-viral agents include interferons (e.g. α-, β-, γ-interferon), cytokines such as tumor necrosis factor (TNF) or interleukins, cell receptors and growth factor antagonists, which can be purified or recombinantly produced.

In some embodiments, the antiviral agent is administered at a dose of less than 3000 mg/day. In some embodiments, the antiviral agent is administered at a dose of about 10 mg/day, about 20 mg/day, about 50 mg/day, about 100 mg/day, about 150 mg/day, about 200 mg/day, about 250 mg/day, about 300 mg/day, about 350 mg/day, about 400 mg/day, about 450 mg/day, about 500 mg/day, about 600 mg/day, about 700 mg/day, about 800 mg/day, about 900 mg/day, about 1000 mg/day, about 1200 mg/day, about 1250 mg/day, about 1400 mg/day, about 1500 mg/day, about 1600 mg/day, about 1750 mg/day, about 1800 mg/day, about 1900 mg/day, about 2000 mg/day, about 2250 mg/day, about 2500 mg/day, about 2750 mg/day, about 3000 mg/day, about 3250 mg/day, about 350 0 mg/day, about 3750 mg/day, about 4000 mg/day, about 4250 mg/day, about 4500 mg/day, about 4750 mg/day, or about 5000 mg/day. In certain embodiments, the antiviral agent is administered at a dose of less than 10 mg/day, less than 20 mg/day, less than 50 mg/day, less than 100 mg/day, less than 150 mg/day, less than 200 mg/day, less than 250 mg/day, less than 300 mg/day, less than 350 mg/day, less than 400 mg/day, less than 450 mg/day, less than 500 mg/day, less than 600 mg/day, less than 700 mg/day, less than 800 mg/day, less than 900 mg/day, less than 1000 mg/day, less than 1200 mg/day, less than 1250 mg/day, less than 1400 mg/day, less than 1500 mg/day, less than 1600 mg/day, less than 1750 mg/day, less than 1800 mg/day, less than 1900 mg/day, less than 2000 mg/day, less than 2250 mg/day, less than 2500 mg/day, less than 2750 mg/day, less than 3000 mg/day, less than 3250 mg/day, less than 3500 mg/day, less than 3750 mg/day, less than 4000 mg/day, less than 4250 mg/day, less than 4500 mg/day, less than 4750 mg/day, or less than 5000 mg/day. In some embodiments, the antiviral agent is administered at a dose of more than 10 mg/day, more than 20 mg/day, more than 50 mg/day, more than 100 mg/day, more than 150 mg/day, more than 200 mg/day, more than 250 mg/day, more than 300 mg/day, more than 350 mg/day, more than 400 mg/day, more than 450 mg/day, more than 500 mg/day, more than 600 mg/day, more than 700 mg/day, more than 800 mg/day, more than 900 mg/day, more than 1000 mg/day, more than 1200 mg/day, more than 1250 mg/day, more than 1400 mg/day, more than 1500 mg/day, more than 1600 mg/day, more than 1750 mg/day, more than 1800 mg/day, more than 1900 mg/day, more than 2000 mg/day, more than 2250 mg/day, more than 2500 mg/day, more than 2750 mg/day, more than 3000 mg/day, more than 3250 mg/day, more than 3500 mg/day, more than 3750 mg/day, more than 4000 mg/day, more than 4250 mg/day, more than 4500 mg/day, more than 4750 mg/day, or more than 5000 mg/day. In certain embodiments, the antiviral agent is administered at a dose of more than 10 mg/day and less than 5000 mg/day. In some embodiments, the antiviral agent is administered at a dose of more than 200 mg/day and less than 1000 mg/day. In certain embodiments, the antiviral agent is administered once a day (q.d, QD.), twice a day (b.id., BID), or thrice a day (t.i.d., TID). In some embodiments, the antiviral agent is administered daily, once a week, twice a week, three times a week, four times a week, or five times a week.

In certain embodiments, the antiviral agent is ganciclovir. In some embodiments, ganciclovir is administered at a total daily dose of 3000 mg/day. In certain embodiments, ganciclovir is administered at a dose of 1000 mg three times a day. In some embodiments, ganciclovir is administered at a dose of about 100 mg/day, about 250 mg/day, about 500 mg/day, about 750 mg/day, about 1000 mg/day, about 1500 mg/day, about 2000 mg/day, about 2500 mg/day, about 3000 mg/day, about 3500 mg/day, or about 4000 mg/day. In certain embodiments, ganciclovir is administered at a dose of less than 100 mg/day, less than 250 mg/day, less than 500 mg/day, less than 750 mg/day, less than 1000 mg/day, less than 1500 mg/day, less than 2000 mg/day, less than 2500 mg/day, less than 3000 mg/day, less than 3500 mg/day, or less than 4000 mg/day. In some embodiments, ganciclovir is administered at a dose of more than 100 mg/day, more than 250 mg/day, more than 500 mg/day, more than 750 mg/day, more than 1000 mg/day, more than 1500 mg/day, more than 2000 mg/day, more than 2500 mg/day, more than 3000 mg/day, more than 3500 mg/day, or more than 4000 mg/day. In certain embodiments, ganciclovir is administered at a dose of more than 500 mg/day and less 4000 mg/day. In some embodiments, ganciclovir is administered at a dose of more than 1000 mg/day and less than 3000 mg/day. In some embodiments, ganciclovir is administered once a day, twice a day, or three times a day. In certain embodiments, ganciclovir is administered once a week, twice a week, three times a week, four times a week, five times a week, or daily. In certain embodiments, the antiviral agent's dose is adjusted to manage side-effects associated with the antiviral in accordance with label instructions.

In some embodiments, the antiviral agent is valganciclovir. In certain embodiments, valganciclovir is administered at a total daily dose of 450 mg/day. In certain embodiments, valganciclovir is administered at a total daily dose of 900 mg/day. In some embodiments, valganciclovir is administered at a dose of 900 mg once a day. In certain embodiments, valganciclovir is administered at a total daily dose of 1800 mg/day. In some embodiments, valganciclovir is administered at a dose of 900 mg twice a day.

In some embodiments, valganciclovir is administered at a dose of about 100 mg/day, about 200 mg/day, about 300 mg/day, about 400 mg/day, about 500 mg/day, about 600 mg/day, about 700 mg/day, about 800 mg/day, about 900 mg/day, about 1000 mg/day, about 1100 mg/day, about 1200 mg/day, about 1300 mg/day, about 1400 mg/day, about 1500 mg/day, about 1600 mg/day, about 1700 mg/day, about 1800 mg/day, about 1900 mg/day, or about 2000 mg/day. In certain embodiments, valganciclovir is administered at a dose of less than 100 mg/day, less than 200 mg/day, less than 300 mg/day, less than 400 mg/day, less than 500 mg/day, less than 600 mg/day, less than 700 mg/day, less than 800 mg/day, less than 900 mg/day, less than 1000 mg/day, less than 1100 mg/day, less than 1200 mg/day, less than 1300 mg/day, less than 1400 mg/day, less than 1500 mg/day, less than 1600 mg/day, less than 1700 mg/day, less than 1800 mg/day, less than 1900 mg/day, or less than 2000 mg/day. In some embodiments, valganciclovir is administered at a dose of more than 100 mg/day, more than 200 mg/day, more than 300 mg/day, more than 400 mg/day, more than 500 mg/day, more than 600 mg/day, more than 700 mg/day, more than 800 mg/day, more than 900 mg/day, more than 1000 mg/day, more than 1100 mg/day, more than 1200 mg/day, more than 1300 mg/day, more than 1400 mg/day, more than 1500 mg/day, more than 1600 mg/day, more than 1700 mg/day, more than 1800 mg/day, more than 1900 mg/day, or more than 2000 mg/day. In certain embodiments, valganciclovir is administered at a dose of more than 100 mg/day and less 2000 mg/day. In some embodiments, valganciclovir is administered at a dose of more than 500 mg/day and less than 1500 mg/day. In some embodiments, valganciclovir is administered once a day, twice a day, or three times a day. In certain embodiments, valganciclovir is administered once a week, twice a week, three times a week, four times a week, five times a week, or daily. In certain embodiments, valganciclovir is administered daily at a dose of about 900 milligrams. In certain embodiments, valganciclovir is administered daily at a dose of about 800 milligrams. In certain embodiments, valganciclovir is administered daily at a dose of about 700 milligrams. In certain embodiments, valganciclovir is administered daily at a dose of about 600 milligrams. In certain embodiments, valganciclovir is administered daily at a dose of about 500 milligrams. In certain embodiments, valganciclovir is administered daily at a dose of about 450 milligrams. In certain embodiments, valganciclovir is administered daily at a dose of about 400 milligrams. In certain embodiments, the valganciclovir is administered daily even on days when no HDACi is administered.

### Dosages of HDACi

The methods described herein allow for the dosing of HDACi at lower doses compared to doses used when the HDACi is deployed as a monotherapy. Such a dosing scheme is advantageous to reduce serious adverse events (SAEs). The SAE reduced can be any one or more of anemia, thrombocytopenia, hypocalcemia, hypophosphatemia, atrial fibrillation, hyperbilirubinemia, and QTcF prolongation. The SAE reduced can be any one or more of nausea, fatigue, anorexia, vomiting, and diarrhea. HDACi prevent maturation of platelets and thus can lead to thrombocytopenia. Dose regimens can be designed to prevent these side-effects by dosing HDACi with short half-lives at levels lower than their maximum tolerated doses. HDACi with short half-lives can be administered at lower levels or in conjunction with a dose holiday or a dose schedule where the HDACi is not administered on certain days of the schedule, such dosages schedules allows for greater platelet maturation and higher levels of platelets. In certain embodiments, an HDACi used in the methods described herein can be administered to an individual at a level that is about 75%, 60%, 50%, 40%, 30%, 20%, or 10% or less of the maximum tolerated dose. In certain embodiments, an HDACi used in the methods described herein can be administered to an individual at a level that is about 50% or less of the maximum tolerated dose. In certain embodiments, an HDACi used in the methods described herein can be administered to an individual at a level that is about 30% or less of the maximum tolerated dose. In certain embodiments, an HDACi used in the methods described herein can be administered to an individual at a level that is about 25% or less of the maximum tolerated dose. In certain embodiments, an HDACi used in the methods described herein can be administered to an individual at a level that is about 20% or less of the maximum tolerated dose. In certain embodiments, an HDACi used in the methods described herein can be administered to an individual at a level that is about 15% or less of the maximum tolerated dose. In certain embodiments, an HDACi used in the methods described herein can be administered to an individual at a level that is about 10% or less of the maximum tolerated dose. In certain embodiments, an HDACi used in the methods described herein can be administered to an individual at a level that is about 5% or less of the maximum tolerated dose. The “maximum tolerated dose” is the highest dose of a drug or treatment that does not cause unacceptable side effects. A maximum tolerated dose can be established experimentally and will generally vary based on the specific PK/PD characteristics of the drug. Generally, the maximum tolerated dose is determined during a clinical trial. In certain embodiments, the maximum tolerated dosage is the approved label dose.

The dosage of an HDACi using the methods described herein can also be determined based upon a probability of developing an adverse event. In certain embodiments, the dosage of HDACi can be determined to avoid grade 3 or 4 adverse events. A grade 3 adverse event is one that is severe but not life threatening. Hospitalization is usually required for a grade 3 adverse event. Examples of grade 3 adverse events include: anemia (Hemoglobin <8.0 g/dL); neutropenia (<1000/mm³ to 500/mm³); thrombocytopenia (<50,000/mm³ to 25,000/mm³); creatinine increase (>3.0×baseline; >3.0 to 6.0×ULN); fatigue (not relieved by rest; limitation of self-care), or nausea/vomiting (6 or more episodes in a 24-hour period). A grade 4 adverse event is one that is life threatening or disabling. Examples of grade 4 adverse events include: anemia (life-threatening); neutropenia (<500/mm³); thrombocytopenia (<25,000/mm³); creatinine increase (>6.0×ULN), or nausea/vomiting (requires parenteral nutrition or other support). In certain embodiments, the dose of HDACi is one with less than about a 50%, 40%, 30%, 25%, 20%, 10% or 5% probability of resulting in a grade 3 adverse event. In certain embodiments, the dose of HDACi is one with less than about a 25%, 20%, 10%, or 5% probability of resulting in a grade 4 adverse event.

The dosage of an HDACi using the methods described herein can be one that is lower than the dose approved for that HDACi when the HDACi is administered or approved as a monotherapy. Romidepsin is approved for use at 14 mg/m² administered IV on days 1, 8, and 15 of a 28-day treatment cycle. At this dosage level Romidepsin has a Grade 3 or 4 adverse effect rate of 35% or 83% according to the approved label. Grade 3 and 4 adverse reactions include anemia and thrombocytopenia. Using the methods described herein the grade 3 or grade 4 adverse effect rate can be reduced to below 35%. Using the methods described herein romidepsin can be administered at a dosage amount of less than about 10, 9, 8, 7, 6, 5, 4, 3, 2, 1.5, or 1 mg/m² using the same route and schedule. In certain embodiments, romidepsin is administered at about 1 mg/m² to about 12 mg/m². In certain embodiments, romidepsin is administered at about 12 mg/m² to about 11 mg/m², about 12 mg/m² to about 10 mg/m², about 12 mg/m² to about 9 mg/m², about 12 mg/m² to about 8 mg/m², about 12 mg/m² to about 7 mg/m², about 12 mg/m² to about 6 mg/m², about 12 mg/m² to about 5 mg/m², about 12 mg/m² to about 4 mg/m², about 12 mg/m² to about 3 mg/m², about 12 mg/m² to about 2 mg/m², about 12 mg/m² to about 1 mg/m², about 11 mg/m² to about 10 mg/m², about 11 mg/m² to about 9 mg/m², about 11 mg/m² to about 8 mg/m², about 11 mg/m² to about 7 mg/m², about 11 mg/m² to about 6 mg/m², about 11 mg/m² to about 5 mg/m², about 11 mg/m² to about 4 mg/m², about 11 mg/m² to about 3 mg/m², about 11 mg/m² to about 2 mg/m², about 11 mg/m² to about 1 mg/m², about 10 mg/m² to about 9 mg/m², about 10 mg/m² to about 8 mg/m², about 10 mg/m to about 7 mg/m, about 10 mg/m to about 6 mg/m², about 10 mg/m² to about 5 mg/m², about 10 mg/m² to about 4 mg/m², about 10 mg/m² to about 3 mg/m², about 10 mg/m² to about 2 mg/m², about 10 mg/m² to about 1 mg/m², about 9 mg/m² to about 8 mg/m², about 9 mg/m² to about 7 mg/m², about 9 mg/m² to about 6 mg/m², about 9 mg/m² to about 5 mg/m², about 9 mg/m² to about 4 mg/m², about 9 mg/m² to about 3 mg/m², about 9 mg/m² to about 2 mg/m², about 9 mg/m² to about 1 mg/m², about 8 mg/m² to about 7 mg/m², about 8 mg/m² to about 6 mg/m², about 8 mg/m² to about 5 mg/m², about 8 mg/m² to about 4 mg/m², about 8 mg/m² to about 3 mg/m², about 8 mg/m² to about 2 mg/m², about 8 mg/m² to about 1 mg/m², about 7 mg/m² to about 6 mg/m², about 7 mg/m² to about 5 mg/m², about 7 mg/m² to about 4 mg/m², about 7 mg/m² to about 3 mg/m², about 7 mg/m² to about 2 mg/m², about 7 mg/m² to about 1 mg/m², about 6 mg/m² to about 5 mg/m², about 6 mg/m² to about 4 mg/m², about 6 mg/m² to about 3 mg/m², about 6 mg/m² to about 2 mg/m², about 6 mg/m² to about 1 mg/m², about 5 mg/m² to about 4 mg/m², about 5 mg/m² to about 3 mg/m², about 5 mg/m² to about 2 mg/m², about 5 mg/m² to about 1 mg/m², about 4 mg/m² to about 3 mg/m², about 4 mg/m² to about 2 mg/m², about 4 mg/m² to about 1 mg/m², about 3 mg/m² to about 2 mg/m², about 3 mg/m² to about 1 mg/m², or about 2 mg/m² to about 1 mg/m². In certain embodiments, romidepsin is administered at about 12 mg/m², about 11 mg/m², about 10 mg/m², about 9 mg/m², about 8 mg/m², about 7 mg/m², about 6 mg/m², about 5 mg/m², about 4 mg/m², about 3 mg/m², about 2 mg/m², or about 1 mg/m². In certain embodiments, romidepsin is administered at least about 12 mg/m², about 11 mg/m², about 10 mg/m², about 9 mg/m², about 8 mg/m², about 7 mg/m², about 6 mg/m², about 5 mg/m², about 4 mg/m², about 3 mg/m², or about 2 mg/m². In certain embodiments, romidepsin is administered at most about 11 mg/m², about 10 mg/m², about 9 mg/m², about 8 mg/m², about 7 mg/m², about 6 mg/m², about 5 mg/m², about 4 mg/m², about 3 mg/m², about 2 mg/m², or about 1 mg/m².

The dosage of an HDACi using the methods described herein can be one that is lower than the dose approved for that HDACi when the HDACi is administered or approved as a monotherapy. Belinostat is approved for use at 1,000 mg/m² administered IV once daily on days 1 to 5 of a 21-day cycle. At this dosage level belinostat has a Grade 3 or 4 adverse effect rate of 47% according to the approved label. Using the methods described herein the grade 3 or grade 4 adverse effect rate can be reduced to below 47%. Using the methods described herein belinostat can be administered at a dosage amount of less than about 1000, 900, 800, 700, 60, 500, 450, 400, 350, 300, 250, 200, 150, or 100 mg/m² using the same route and schedule. In certain embodiments, belinostat is administered at about 50 mg/m² to about 1,000 mg/m². In certain embodiments, belinostat is administered at about 1,000 mg/m² to about 900 mg/m², about 1,000 mg/m² to about 800 mg/m², about 1,000 mg/m² to about 700 mg/m², about 1,000 mg/m² to about 600 mg/m², about 1,000 mg/m² to about 500 mg/m², about 1,000 mg/m² to about 400 mg/m², about 1,000 mg/m² to about 300 mg/m², about 1,000 mg/m² to about 200 mg/m², about 1,000 mg/m² to about 100 mg/m², about 1,000 mg/m² to about 50 mg/m², about 900 mg/m² to about 800 mg/m², about 900 mg/m² to about 700 mg/m², about 900 mg/m² to about 600 mg/m², about 900 mg/m² to about 500 mg/m², about 900 mg/m² to about 400 mg/m², about 900 mg/m² to about 300 mg/m², about 900 mg/m² to about 200 mg/m², about 900 mg/m² to about 100 mg/m², about 900 mg/m² to about 50 mg/m², about 800 mg/m² to about 700 mg/m², about 800 mg/m² to about 600 mg/m², about 800 mg/m² to about 500 mg/m², about 800 mg/m² to about 400 mg/m², about 800 mg/m² to about 300 mg/m², about 800 mg/m² to about 200 mg/m², about 800 mg/m² to about 100 mg/m², about 800 mg/m² to about 50 mg/m², about 700 mg/m² to about 600 mg/m², about 700 mg/m² to about 500 mg/m², about 700 mg/m² to about 400 mg/m², about 700 mg/m² to about 300 mg/m², about 700 mg/m² to about 200 mg/m², about 700 mg/m² to about 100 mg/m², about 700 mg/m² to about 50 mg/m², about 600 mg/m² to about 500 mg/m², about 600 mg/m² to about 400 mg/m², about 600 mg/m² to about 300 mg/m², about 600 mg/m² to about 200 mg/m², about 600 mg/m² to about 100 mg/m², about 600 mg/m² to about 50 mg/m², about 500 mg/m² to about 400 mg/m², about 500 mg/m² to about 300 mg/m², about 500 mg/m² to about 200 mg/m², about 500 mg/m² to about 100 mg/m², about 500 mg/m² to about 50 mg/m², about 400 mg/m² to about 300 mg/m², about 400 mg/m² to about 200 mg/m², about 400 mg/m² to about 100 mg/m², about 400 mg/m² to about 50 mg/m², about 300 mg/m² to about 200 mg/m², about 300 mg/m² to about 100 mg/m², about 300 mg/m² to about 50 mg/m², about 200 mg/m² to about 100 mg/m², about 200 mg/m² to about 50 mg/m², or about 100 mg/m² to about 50 mg/m². In certain embodiments, belinostat is administered at about 1,000 mg/m², about 900 mg/m², about 800 mg/m², about 700 mg/m², about 600 mg/m², about 500 mg/m², about 400 mg/m², about 300 mg/m², about 200 mg/m², about 100 mg/m², or about 50 mg/m². In certain embodiments, belinostat is administered at least about 1,000 mg/m², about 900 mg/m², about 800 mg/m², about 700 mg/m², about 600 mg/m², about 500 mg/m², about 400 mg/m², about 300 mg/m², about 200 mg/m², or about 100 mg/m². In certain embodiments, belinostat is administered at most about 900 mg/m², about 800 mg/m², about 700 mg/m², about 600 mg/m², about 500 mg/m², about 400 mg/m², about 300 mg/m², about 200 mg/m², about 100 mg/m², or about 50 mg/m². The dosage of an HDACi using the methods described herein can be one that is lower than the dose approved for that HDACi when the HDACi is approved as a monotherapy. Vorinostat is approved for use at 400 q.d. with food or 300 q.d. without food. At this dosage level Using the methods described herein vorinostat can be administered at a dosage amount of less than about 400, 300, 200, 100, or 50 q.d. In certain embodiments, vorinostat is administered at about 50 mg to about 400 mg. In certain embodiments, vorinostat is administered at about 400 mg to about 350 mg, about 400 mg to about 300 mg, about 400 mg to about 250 mg, about 400 mg to about 200 mg, about 400 mg to about 150 mg, about 400 mg to about 100 mg, about 400 mg to about 50 mg, about 350 mg to about 300 mg, about 350 mg to about 250 mg, about 350 mg to about 200 mg, about 350 mg to about 150 mg, about 350 mg to about 100 mg, about 350 mg to about 50 mg, about 300 mg to about 250 mg, about 300 mg to about 200 mg, about 300 mg to about 150 mg, about 300 mg to about 100 mg, about 300 mg to about 50 mg, about 250 mg to about 200 mg, about 250 mg to about 150 mg, about 250 mg to about 100 mg, about 250 mg to about 50 mg, about 200 mg to about 150 mg, about 200 mg to about 100 mg, about 200 mg to about 50 mg, about 150 mg to about 100 mg, about 150 mg to about 50 mg, or about 100 mg to about 50 mg. In certain embodiments, vorinostat is administered at about 400 mg, about 350 mg, about 300 mg, about 250 mg, about 200 mg, about 150 mg, about 100 mg, or about 50 mg. In certain embodiments, vorinostat is administered at least about 400 mg, about 350 mg, about 300 mg, about 250 mg, about 200 mg, about 150 mg, or about 100 mg. In certain embodiments, vorinostat is administered at most about 350 mg, about 300 mg, about 250 mg, about 200 mg, about 150 mg, about 100 mg, or about 50 mg. In certain embodiments, vorinostat is dosed on only certain days of a schedule. This schedule can be any one or more of 1, 2, 3, 4, 5, 6, or 7 days of a one-week schedule.

The dosage of an HDACi using the methods described herein can be one that is lower than the dose approved or recommended for that HDACi when the HDACi is approved as a monotherapy. Chidamide when dosed at 30 mg twice weekly exhibits an SAE rate of 5% with an objective response rate of 28%. However, the most common side-effects observed were hematological side effects such as thrombocytopenia, neutropenia, and leukopenia. Using the methods described herein the grade 3 or grade 4 adverse effect rate can be reduced to below 28%. Using the methods described herein hematologic side effects may be reduced, the doses of chidamide can be reduced below 30 mg twice weekly. In certain embodiments, chidamide is administered at about 5 mg to about 30 mg. In certain embodiments, chidamide is administered at about 30 mg to about 25 mg, about 30 mg to about 20 mg, about 30 mg to about 17.5 mg, about 30 mg to about 15 mg, about 30 mg to about 12.5 mg, about 30 mg to about 10 mg, about 30 mg to about 7.5 mg, about 30 mg to about 5 mg, about 25 mg to about 20 mg, about 25 mg to about 17.5 mg, about 25 mg to about 15 mg, about 25 mg to about 12.5 mg, about 25 mg to about 10 mg, about 25 mg to about 7.5 mg, about 25 mg to about 5 mg, about 20 mg to about 17.5 mg, about 20 mg to about 15 mg, about 20 mg to about 12.5 mg, about 20 mg to about 10 mg, about 20 mg to about 7.5 mg, about 20 mg to about 5 mg, about 17.5 mg to about 15 mg, about 17.5 mg to about 12.5 mg, about 17.5 mg to about 10 mg, about 17.5 mg to about 7.5 mg, about 17.5 mg to about 5 mg, about 15 mg to about 12.5 mg, about 15 mg to about 10 mg, about 15 mg to about 7.5 mg, about 15 mg to about 5 mg, about 12.5 mg to about 10 mg, about 12.5 mg to about 7.5 mg, about 12.5 mg to about 5 mg, about 10 mg to about 7.5 mg, about 10 mg to about 5 mg, or about 7.5 mg to about 5 mg. In certain embodiments, chidamide is administered at about 30 mg, about 25 mg, about 20 mg, about 17.5 mg, about 15 mg, about 12.5 mg, about 10 mg, about 7.5 mg, or about 5 mg. In certain embodiments, chidamide is administered at least about 30 mg, about 25 mg, about 20 mg, about 17.5 mg, about 15 mg, about 12.5 mg, about 10 mg, or about 7.5 mg. In certain embodiments, chidamide is administered at most about 25 mg, about 20 mg, about 17.5 mg, about 15 mg, about 12.5 mg, about 10 mg, about 7.5 mg, or about 5 mg. Overall dosages can be lowered using the methods described herein, resulting in lower dosage levels, longer time periods between dosages or a combination of the two. Chidamide can be administered at any of these doses daily, every other day, twice, three times, four times, or five times weekly. In certain embodiments, chidamide is dosed on only certain days of a schedule. This schedule can be any one or more of 1, 2, 3, 4, 5, 6, or 7 days of a one-week schedule. In certain embodiments, chidamide is dosed on only certain days of a schedule. This schedule can be any one or more of 1, 2, 3, 4, 5, 6, or 7 days of a one-week schedule.

The dosage of an HDACi using the methods described herein can be one that is lower than the dose approved or recommended for that HDACi when the HDACi is approved as a monotherapy. In certain embodiments, entinostat is administered at about 0.25 mg once weekly to about 5 mg once weekly. In certain embodiments, entinostat is administered at about 5 mg once weekly to about 4 mg once weekly, about 5 mg once weekly to about 3 mg once weekly, about 5 mg once weekly to about 2 mg once weekly, about 5 mg once weekly to about 1 mg once weekly, about 5 mg once weekly to about 0.5 mg once weekly, about 5 mg once weekly to about 0.25 mg once weekly, about 4 mg once weekly to about 3 mg once weekly, about 4 mg once weekly to about 2 mg once weekly, about 4 mg once weekly to about 1 mg once weekly, about 4 mg once weekly to about 0.5 mg once weekly, about 4 mg once weekly to about 0.25 mg once weekly, about 3 mg once weekly to about 2 mg once weekly, about 3 mg once weekly to about 1 mg once weekly, about 3 mg once weekly to about 0.5 mg once weekly, about 3 mg once weekly to about 0.25 mg once weekly, about 2 mg once weekly to about 1 mg once weekly, about 2 mg once weekly to about 0.5 mg once weekly, about 2 mg once weekly to about 0.25 mg once weekly, about 1 mg once weekly to about 0.5 mg once weekly, about 1 mg once weekly to about 0.25 mg once weekly, or about 0.5 mg once weekly to about 0.25 mg once weekly. In certain embodiments, entinostat is administered at about 5 mg once weekly, about 4 mg once weekly, about 3 mg once weekly, about 2 mg once weekly, about 1 mg once weekly, about 0.5 mg once weekly, or about 0.25 mg once weekly. In certain embodiments, entinostat is administered at least about 5 mg once weekly, about 4 mg once weekly, about 3 mg once weekly, about 2 mg once weekly, about 1 mg once weekly, or about 0.5 mg once weekly. In certain embodiments, entinostat is administered at most about 4 mg once weekly, about 3 mg once weekly, about 2 mg once weekly, about 1 mg once weekly, about 0.5 mg once weekly, or about 0.25 mg once weekly. Overall dosages can be lowered using the methods described herein, resulting in lower dosage levels, longer time periods between dosages or a combination of the two. Entinostat can be administered at any of these doses daily, every other day, twice, three times, four times, or five times weekly. In certain embodiments, entinostat is dosed on only certain days of a schedule. This schedule can be any one or more of 1, 2, 3, 4, 5, 6, or 7 days of a one-week schedule. In certain embodiments, entinostat is dosed on only certain days of a schedule. This schedule can be any one or more of 1, 2, 3, 4, 5, 6, or 7 days of a one-week schedule.

The dosage of an HDACi using the methods described herein can be one that is lower than the dose approved or recommended for that HDACi when the HDACi is approved as a monotherapy. In certain embodiments, quisinostat is administered at about 1 mg every other day to about 12 mg every other day. In certain embodiments, quisinostat is administered at about 12 mg every other day to about 11 mg every other day, about 12 mg every other day to about 10 mg every other day, about 12 mg every other day to about 9 mg every other day, about 12 mg every other day to about 8 mg every other day, about 12 mg every other day to about 7 mg every other day, about 12 mg every other day to about 6 mg every other day, about 12 mg every other day to about 5 mg every other day, about 12 mg every other day to about 4 mg every other day, about 12 mg every other day to about 3 mg every other day, about 12 mg every other day to about 2 mg every other day, about 12 mg every other day to about 1 mg every other day, about 11 mg every other day to about 10 mg every other day, about 11 mg every other day to about 9 mg every other day, about 11 mg every other day to about 8 mg every other day, about 11 mg every other day to about 7 mg every other day, about 11 mg every other day to about 6 mg every other day, about 11 mg every other day to about 5 mg every other day, about 11 mg every other day to about 4 mg every other day, about 11 mg every other day to about 3 mg every other day, about 11 mg every other day to about 2 mg every other day, about 11 mg every other day to about 1 mg every other day, about 10 mg every other day to about 9 mg every other day, about 10 mg every other day to about 8 mg every other day, about 10 mg every other day to about 7 mg every other day, about 10 mg every other day to about 6 mg every other day, about 10 mg every other day to about 5 mg every other day, about 10 mg every other day to about 4 mg every other day, about 10 mg every other day to about 3 mg every other day, about 10 mg every other day to about 2 mg every other day, about 10 mg every other day to about 1 mg every other day, about 9 mg every other day to about 8 mg every other day, about 9 mg every other day to about 7 mg every other day, about 9 mg every other day to about 6 mg every other day, about 9 mg every other day to about 5 mg every other day, about 9 mg every other day to about 4 mg every other day, about 9 mg every other day to about 3 mg every other day, about 9 mg every other day to about 2 mg every other day, about 9 mg every other day to about 1 mg every other day, about 8 mg every other day to about 7 mg every other day, about 8 mg every other day to about 6 mg every other day, about 8 mg every other day to about 5 mg every other day, about 8 mg every other day to about 4 mg every other day, about 8 mg every other day to about 3 mg every other day, about 8 mg every other day to about 2 mg every other day, about 8 mg every other day to about 1 mg every other day, about 7 mg every other day to about 6 mg every other day, about 7 mg every other day to about 5 mg every other day, about 7 mg every other day to about 4 mg every other day, about 7 mg every other day to about 3 mg every other day, about 7 mg every other day to about 2 mg every other day, about 7 mg every other day to about 1 mg every other day, about 6 mg every other day to about 5 mg every other day, about 6 mg every other day to about 4 mg every other day, about 6 mg every other day to about 3 mg every other day, about 6 mg every other day to about 2 mg every other day, about 6 mg every other day to about 1 mg every other day, about 5 mg every other day to about 4 mg every other day, about 5 mg every other day to about 3 mg every other day, about 5 mg every other day to about 2 mg every other day, about 5 mg every other day to about 1 mg every other day, about 4 mg every other day to about 3 mg every other day, about 4 mg every other day to about 2 mg every other day, about 4 mg every other day to about 1 mg every other day, about 3 mg every other day to about 2 mg every other day, about 3 mg every other day to about 1 mg every other day, or about 2 mg every other day to about 1 mg every other day. In certain embodiments, quisinostat is administered at about 12 mg every other day, about 11 mg every other day, about 10 mg every other day, about 9 mg every other day, about 8 mg every other day, about 7 mg every other day, about 6 mg every other day, about 5 mg every other day, about 4 mg every other day, about 3 mg every other day, about 2 mg every other day, or about 1 mg every other day. In certain embodiments, quisinostat is administered at least about 12 mg every other day, about 11 mg every other day, about 10 mg every other day, about 9 mg every other day, about 8 mg every other day, about 7 mg every other day, about 6 mg every other day, about 5 mg every other day, about 4 mg every other day, about 3 mg every other day, or about 2 mg every other day. In certain embodiments, quisinostat is administered at most about 11 mg every other day, about 10 mg every other day, about 9 mg every other day, about 8 mg every other day, about 7 mg every other day, about 6 mg every other day, about 5 mg every other day, about 4 mg every other day, about 3 mg every other day, about 2 mg every other day, or about 1 mg every other day. Overall dosages can be lowered using the methods described herein, resulting in lower dosage levels, longer time periods between dosages or a combination of the two. Quisinostat can be administered at any of these doses daily, every other day, twice, three times, four times, or five times weekly. In certain embodiments, entinostat is dosed on only certain days of a schedule. This schedule can be any one or more of 1, 2, 3, 4, 5, 6, or 7 days of a one-week schedule. In certain embodiments, quisinostat is dosed on only certain days of a schedule. This schedule can be any one or more of 1, 2, 3, 4, 5, 6, or 7 days of a one-week schedule.

The dosage of an HDACi using the methods described herein can be one that is lower than the dose approved or recommended for that HDACi when the HDACi is approved as a monotherapy. In certain embodiments, mocetinostat is administered at about 1 mg 3 times a week to about 70 mg 3 times a week. In certain embodiments, mocetinostat is administered at about 70 mg 3 times a week to about 60 mg 3 times a week, about 70 mg 3 times a week to about 50 mg 3 times a week, about 70 mg 3 times a week to about 40 mg 3 times a week, about 70 mg 3 times a week to about 30 mg 3 times a week, about 70 mg 3 times a week to about 20 mg 3 times a week, about 70 mg 3 times a week to about 10 mg 3 times a week, about 70 mg 3 times a week to about 5 mg 3 times a week, about 70 mg 3 times a week to about 4 mg 3 times a week, about 70 mg 3 times a week to about 3 mg 3 times a week, about 70 mg 3 times a week to about 2 mg 3 times a week, about 70 mg 3 times a week to about 1 mg 3 times a week, about 60 mg 3 times a week to about 50 mg 3 times a week, about 60 mg 3 times a week to about 40 mg 3 times a week, about 60 mg 3 times a week to about 30 mg 3 times a week, about 60 mg 3 times a week to about 20 mg 3 times a week, about 60 mg 3 times a week to about 10 mg 3 times a week, about 60 mg 3 times a week to about 5 mg 3 times a week, about 60 mg 3 times a week to about 4 mg 3 times a week, about 60 mg 3 times a week to about 3 mg 3 times a week, about 60 mg 3 times a week to about 2 mg 3 times a week, about 60 mg 3 times a week to about 1 mg 3 times a week, about 50 mg 3 times a week to about 40 mg 3 times a week, about 50 mg 3 times a week to about 30 mg 3 times a week, about 50 mg 3 times a week to about 20 mg 3 times a week, about 50 mg 3 times a week to about 10 mg 3 times a week, about 50 mg 3 times a week to about 5 mg 3 times a week, about 50 mg 3 times a week to about 4 mg 3 times a week, about 50 mg 3 times a week to about 3 mg 3 times a week, about 50 mg 3 times a week to about 2 mg 3 times a week, about 50 mg 3 times a week to about 1 mg 3 times a week, about 40 mg 3 times a week to about 30 mg 3 times a week, about 40 mg 3 times a week to about 20 mg 3 times a week, about 40 mg 3 times a week to about 10 mg 3 times a week, about 40 mg 3 times a week to about 5 mg 3 times a week, about 40 mg 3 times a week to about 4 mg 3 times a week, about 40 mg 3 times a week to about 3 mg 3 times a week, about 40 mg 3 times a week to about 2 mg 3 times a week, about 40 mg 3 times a week to about 1 mg 3 times a week, about 30 mg 3 times a week to about 20 mg 3 times a week, about 30 mg 3 times a week to about 10 mg 3 times a week, about 30 mg 3 times a week to about 5 mg 3 times a week, about 30 mg 3 times a week to about 4 mg 3 times a week, about 30 mg 3 times a week to about 3 mg 3 times a week, about 30 mg 3 times a week to about 2 mg 3 times a week, about 30 mg 3 times a week to about 1 mg 3 times a week, about 20 mg 3 times a week to about 10 mg 3 times a week, about 20 mg 3 times a week to about 5 mg 3 times a week, about 20 mg 3 times a week to about 4 mg 3 times a week, about 20 mg 3 times a week to about 3 mg 3 times a week, about 20 mg 3 times a week to about 2 mg 3 times a week, about 20 mg 3 times a week to about 1 mg 3 times a week, about 10 mg 3 times a week to about 5 mg 3 times a week, about 10 mg 3 times a week to about 4 mg 3 times a week, about 10 mg 3 times a week to about 3 mg 3 times a week, about 10 mg 3 times a week to about 2 mg 3 times a week, about 10 mg 3 times a week to about 1 mg 3 times a week, about 5 mg 3 times a week to about 4 mg 3 times a week, about 5 mg 3 times a week to about 3 mg 3 times a week, about 5 mg 3 times a week to about 2 mg 3 times a week, about 5 mg 3 times a week to about 1 mg 3 times a week, about 4 mg 3 times a week to about 3 mg 3 times a week, about 4 mg 3 times a week to about 2 mg 3 times a week, about 4 mg 3 times a week to about 1 mg 3 times a week, about 3 mg 3 times a week to about 2 mg 3 times a week, about 3 mg 3 times a week to about 1 mg 3 times a week, or about 2 mg 3 times a week to about 1 mg 3 times a week. In certain embodiments, mocetinostat is administered at about 70 mg 3 times a week, about 60 mg 3 times a week, about 50 mg 3 times a week, about 40 mg 3 times a week, about 30 mg 3 times a week, about 20 mg 3 times a week, about 10 mg 3 times a week, about 5 mg 3 times a week, about 4 mg 3 times a week, about 3 mg 3 times a week, about 2 mg 3 times a week, or about 1 mg 3 times a week. In certain embodiments, mocetinostat is administered at least about 70 mg 3 times a week, about 60 mg 3 times a week, about 50 mg 3 times a week, about 40 mg 3 times a week, about 30 mg 3 times a week, about 20 mg 3 times a week, about 10 mg 3 times a week, about 5 mg 3 times a week, about 4 mg 3 times a week, about 3 mg 3 times a week, or about 2 mg 3 times a week. In certain embodiments, mocetinostat is administered at most about 60 mg 3 times a week, about 50 mg 3 times a week, about 40 mg 3 times a week, about 30 mg 3 times a week, about 20 mg 3 times a week, about 10 mg 3 times a week, about 5 mg 3 times a week, about 4 mg 3 times a week, about 3 mg 3 times a week, about 2 mg 3 times a week, or about 1 mg 3 times a week. Overall dosages can be lowered using the methods described herein, resulting in lower dosage levels, longer time periods between dosages or a combination of the two. Mocetinostat can be administered at any of these doses daily, every other day, twice, three times, four times, or five times weekly. In certain embodiments, entinostat is dosed on only certain days of a schedule. This schedule can be any one or more of 1, 2, 3, 4, 5, 6, or 7 days of a one-week schedule.

### Methods and Compositions

In one aspect, provided herein are methods for treating and/or preventing a herpesvirus associated condition. In some embodiments, the condition is associated with a latent viral infection. In certain embodiments, the herpesvirus associated condition is a cancer. In certain embodiments, the cancer is associated with infection by the Epstein—Barr virus. In certain embodiments, the cancer is associated with infection by a Herpes simplex virus. In certain embodiments, the cancer is associated with infection by a cytomegalovirus. In certain embodiments, the methods comprise administering a viral inducing agent (e.g., an HDAC inhibitor) and an antiviral agent. In some embodiments, the methods comprise administering an HDAC inhibitor and an antiviral agent. In certain embodiments, the HDAC inhibitor and the antiviral agent are co-formulated. In certain embodiments, the antiviral agent is administered daily while the HDACi is administered on only certain days. In certain embodiments, the HDACi is nanatinostat. In certain embodiments, the HDACi is administered with food or another nutritional supplement. In certain embodiments, the nanatinostat is administered with food or another nutritional supplement.

In some embodiments, the compositions are administered in an intermittent manner. In certain embodiments, this allows for a “dose-hold” or a “structured treatment interruption,” which allows for the management of negative side-effects. In certain embodiments, the HDACi and anti-viral agent are administered for at least one, two, three, four, or five days, of the schedule, and no HDACi is administered for one, two, three, four, or five days of the schedule. In certain embodiments, the schedule is a week, and is repeated for 1, 2, 3, 4, 5, 6, 7, 8, 9, 10 or more times. In certain embodiments, the antiviral is administered on every day of the schedule.

In certain embodiments, the dose schedule is a week, and an HDACi is administered for 1 day of the dose schedule. In certain embodiments, the dose schedule is a week, and an HDACi is administered for 2 days of the dose schedule. In certain embodiments, the dose schedule is a week, and an HDACi is administered for 3 days of the dose schedule. In certain embodiments, the dose schedule is a week, and an HDACi is administered for 4 days of the dose schedule. In certain embodiments, the dose schedule is a week, and an HDACi is administered for 5 days of the dose schedule. In certain embodiments, the dose schedule is a week, and an HDACi is administered for 6 days of the dose schedule. In certain embodiments, the HDACi has an elimination half-life of less than 34 hours. In certain embodiments, the HDACi has an elimination half-life of less than 20 hours. In certain embodiments, the HDACi has an elimination half-life of less than 12 hours. In certain embodiments, the HDACi has an elimination half-life of less than 6 hours. In certain embodiments, the HDACi is administered QD and 30 milligrams. In certain embodiments, the HDACi is administered QD at 20 milligrams. In certain embodiments, the HDACi is administered QD at 15 milligrams. In certain embodiments, the HDACi is administered QD at 10 milligrams. In certain embodiments, the HDACi is administered BID at 15 milligrams. In certain embodiments, the HDACi is administered BID at 10 milligrams. In certain embodiments, the HDACi is administered BID at 5 milligrams. In certain embodiments, an antiviral is administered daily during the schedule. In certain embodiments, the antiviral is valganciclovir, and the valganciclovir is administered at a dose of 900 milligrams or 450 milligrams.

In certain embodiments, the dose schedule is a week, and an HDACi is administered for 1 day followed by 6 days of no HDACi treatment. In certain embodiments, the dose schedule is a week, and an HDACi is administered for 2 days followed by 5 days of no HDACi treatment. In certain embodiments, the dose schedule is a week, and an HDACi is administered for 3 days followed by 4 days of no HDACi treatment. In certain embodiments, the dose schedule is a week, and an HDACi is administered for 4 days followed by 3 days of no HDACi treatment. In certain embodiments, the dose schedule is a week, and an HDACi is administered for 5 days followed by 2 days of no HDACi treatment. In certain embodiments, the dose schedule is a week, and an HDACi is administered for 6 days followed by 1 day of no HDACi treatment. In certain embodiments, the dose schedule is a week, and an HDACi is administered every other day on days 1, 2, 5, and 7. In certain embodiments, the dose schedule is a week, and an HDACi is administered every other day on days 2, 4, and 6. In certain embodiments, the HDACi has an elimination half-life of less than 34 hours. In certain embodiments, the HDACi has an elimination half-life of less than 20 hours. In certain embodiments, the HDACi has an elimination half-life of less than 12 hours. In certain embodiments, the HDACi has an elimination half-life of less than 6 hours. In certain embodiments, an antiviral is administered daily during the schedule. In certain embodiments, the antiviral is valganciclovir, and the valganciclovir is administered at a dose of 900 milligrams or 450 milligrams.

In certain embodiments, the dose schedule is a week, and nanatinostat is administered for 1 day of the dose schedule. In certain embodiments, the dose schedule is a week, and nanatinostat is administered for 2 days of the dose schedule. In certain embodiments, the dose schedule is a week, and nanatinostat is administered for 3 days of the dose schedule. In certain embodiments, the dose schedule is a week, and nanatinostat is administered for 4 days of the dose schedule. In certain embodiments, the dose schedule is a week, and nanatinostat is administered for 5 days of the dose schedule. In certain embodiments, the dose schedule is a week, and nanatinostat is administered for 6 days of the dose schedule. In certain embodiments, an antiviral is administered daily during the schedule. In certain embodiments, the antiviral is valganciclovir, and the valganciclovir is administered at a dose of 900 milligrams or 450 milligrams.

In certain embodiments, the dose schedule is a week, and nanatinostat is administered for 1 day followed by 6 days of no nanatinostat treatment. In certain embodiments, the dose schedule is a week, and nanatinostat is administered for 2 days followed by 5 days of no nanatinostat treatment. In certain embodiments, the dose schedule is a week, and nanatinostat is administered for 3 days followed by 4 days of no nanatinostat treatment. In certain embodiments, the dose schedule is a week, and nanatinostat is administered for 4 days followed by 3 days of no nanatinostat treatment. In certain embodiments, the dose schedule is a week, and nanatinostat is administered for 5 days followed by 2 days of no nanatinostat treatment. In certain embodiments, the dose schedule is a week, and nanatinostat is administered for 6 days followed by 1 day of no nanatinostat treatment. In certain embodiments, the dose schedule is a week, and nanatinostat is administered every other day on days 1, 2, 5, and 7. In certain embodiments, the dose schedule is a week, and nanatinostat is administered every other day on days 2, 4, and 6. In certain embodiments, the nanatinostat is administered QD and 30 milligrams. In certain embodiments, the nanatinostat is administered QD at 20 milligrams. In certain embodiments, the nanatinostat is administered QD at 15 milligrams. In certain embodiments, the nanatinostat is administered QD at 10 milligrams. In certain embodiments, the nanatinostat is administered BID at 15 milligrams. In certain embodiments, the nanatinostat is administered BID at 10 milligrams. In certain embodiments, the nanatinostat is administered BID at 5 milligrams. In certain embodiments, an antiviral is administered daily during the schedule. In certain embodiments, the antiviral is valganciclovir, and the valganciclovir is administered at a dose of 900 milligrams or 450 milligrams.

In certain embodiments, the dose schedule is a week, and nanatinostat is administered 20 mg QD for 1 day followed by 6 days of no nanatinostat treatment. In certain embodiments, the dose schedule is a week, and nanatinostat is administered 20 mg QD for 2 days followed by 5 days of no nanatinostat treatment. In certain embodiments, the dose schedule is a week, and nanatinostat is administered 20 mg QD for 3 days followed by 4 days of no nanatinostat treatment. In certain embodiments, the dose schedule is a week, and nanatinostat is administered 20 mg QD for 4 days followed by 3 days of no HDACi treatment. In certain embodiments, the dose schedule is a week, and nanatinostat is administered 20 mg QD for 5 days followed by 2 days of no nanatinostat treatment. In certain embodiments, the dose schedule is a week, and nanatinostat is administered 20 mg QD for 6 days followed by 1 day of no nanatinostat treatment. In certain embodiments, the dose schedule is a week, and nanatinostat is administered every other day on days 1, 2, 5, and 7. In certain embodiments, the dose schedule is a week, and nanatinostat is administered every other day on days 2, 4, and 6. In certain embodiments, an antiviral is administered daily during the schedule. In certain embodiments, the antiviral is valganciclovir, and the valganciclovir is administered at a dose of 900 milligrams or 450 milligrams.

In certain embodiments, the dose schedule is a week, and nanatinostat is administered 10 mg QD for 1 day followed by 6 days of no nanatinostat treatment. In certain embodiments, the dose schedule is a week, and nanatinostat is administered 10 mg QD for 2 days followed by 5 days of no nanatinostat treatment. In certain embodiments, the dose schedule is a week, and nanatinostat is administered 10 mg QD for 3 days followed by 4 days of no nanatinostat treatment. In certain embodiments, the dose schedule is a week, and nanatinostat is administered 10 mg QD for 4 days followed by 3 days of no HDACi treatment. In certain embodiments, the dose schedule is a week, and nanatinostat is administered 10 mg QD for 5 days followed by 2 days of no nanatinostat treatment. In certain embodiments, the dose schedule is a week, and nanatinostat is administered 10 mg QD for 6 days followed by 1 day of no nanatinostat treatment. In certain embodiments, the dose schedule is a week, and nanatinostat is administered every other day on days 1, 2, 5, and 7. In certain embodiments, the dose schedule is a week, and nanatinostat is administered every other day on days 2, 4, and 6. In certain embodiments, an antiviral is administered daily during the schedule. In certain embodiments, the antiviral is valganciclovir, and the valganciclovir is administered at a dose of 900 milligrams or 450 milligrams.

In certain embodiments, the dose schedule is a week, and nanatinostat is administered 10 mg BID for 1 day followed by 6 days of no nanatinostat treatment. In certain embodiments, the dose schedule is a week, and nanatinostat is administered 10 mg BID for 2 days followed by 5 days of no nanatinostat treatment. In certain embodiments, the dose schedule is a week, and nanatinostat is administered 10 mg BID for 3 days followed by 4 days of no nanatinostat treatment. In certain embodiments, the dose schedule is a week, and nanatinostat is administered 10 mg BID for 4 days followed by 3 days of no HDACi treatment. In certain embodiments, the dose schedule is a week, and nanatinostat is administered 10 mg BID for 5 days followed by 2 days of no nanatinostat treatment. In certain embodiments, the dose schedule is a week, and nanatinostat is administered 10 mg BID for 6 days followed by 1 day of no nanatinostat treatment. In certain embodiments, the dose schedule is a week, and nanatinostat is administered every other day on days 1, 2, 5, and 7. In certain embodiments, the dose schedule is a week, and nanatinostat is administered every other day on days 2, 4, and 6. In certain embodiments, an antiviral is administered daily during the schedule. In certain embodiments, the antiviral is valganciclovir, and the valganciclovir is administered at a dose of 900 milligrams or 450 milligrams.

In certain embodiments, the dose schedule is a week, and nanatinostat is administered 5 mg BID for 1 day followed by 6 days of no nanatinostat treatment. In certain embodiments, the dose schedule is a week, and nanatinostat is administered 5 mg BID for 2 days followed by 5 days of no nanatinostat treatment. In certain embodiments, the dose schedule is a week, and nanatinostat is administered 5 mg BID for 3 days followed by 4 days of no nanatinostat treatment. In certain embodiments, the dose schedule is a week, and nanatinostat is administered 5 mg BID for 4 days followed by 3 days of no HDACi treatment. In certain embodiments, the dose schedule is a week, and nanatinostat is administered 5 mg BID for 5 days followed by 2 days of no nanatinostat treatment. In certain embodiments, the dose schedule is a week, and nanatinostat is administered 5 mg BID for 6 days followed by 1 day of no nanatinostat treatment. In certain embodiments, the dose schedule is a week, and nanatinostat is administered every other day on days 1, 2, 5, and 7. In certain embodiments, the dose schedule is a week, and nanatinostat is administered every other day on days 2, 4, and 6. In certain embodiments, an antiviral is administered daily during the schedule. In certain embodiments, the antiviral is valganciclovir, and the valganciclovir is administered at a dose of 900 milligrams or 450 milligrams.

The HDACi can be given with food or a meal, or a type of nutritional supplement. In certain embodiments, nanatinostat is given with food or a meal or a type of nutritional supplement. Dosing the HDACi with food can be combined with the above-mentioned schedules to further increase the Cmax and bioavailability of the HDACi or nanatinostat.

Also envisioned herein is dose packaging to efficiently and easily implement the dose schedule mentioned above. The packaging can be, for example, a blister pack or other sealable packing that allows the HDACi and antiviral doses for any given day to be accessed. In certain embodiments, the HDACi and antiviral agent can be packaged so that the formulations of each are separate (e.g., one day's dose comprises HDACi and antiviral agent separated. In certain embodiments, the packing can comprise co-formulated HDACi and antiviral agent. In certain embodiments, the packing can comprise co-formulated nanatinostat and valganciclovir. When such packaging is utilized with the schedules disclosed herein, a day when both HDACi and antiviral agent are to be administered the packing comprise a single oral dosage form that comprises both HDACi and antiviral agent; and a day when only an antiviral agent is to be administered the packing can comprise antiviral agent without HDACi. When such packaging is utilized with the schedules disclosed herein, a day when both nanatinostat and valganciclovir are to be administered the packing comprise a single oral dosage form that comprises both nanatinostat and valganciclovir; and a day when only valganciclovir is to be administered the packing can comprise valganciclovir without nanatinostat.

The schedules described herein can be administered to certain patients with HDACi or antiviral side effects. In certain embodiments, the methods described herein encompass selecting a patent with thrombocytopenia. In certain embodiments, the methods and HDACi compositions described herein are for use in a patent with thrombocytopenia. Thrombocytopenia is generally defined as a platelet count below 150,000 platelets per microliter. In certain embodiments, the patient can be selected for treatment by the methods herein with a platelet count of below about 50,000; 75,000; 100,000, or 125,000 platelets per microliter. In certain embodiments, the methods do not encompass a set schedule of HDACi administration, but further monitoring for resolution of thrombocytopenia before retreatment with HDACi.

The schedules described herein can be administered to certain patients with HDACi or antiviral side effects. In certain embodiments, the methods described herein encompass selecting a patent with high creatinine levels or another marker of impaired kidney function. In certain embodiments, the methods and HDACi compositions described herein are for use in a patent with high creatinine levels or another marker of impaired kidney function. A serum creatinine level that exceeds 1.1 mg/dL for a woman or 1.3 mg/dL for a man is generally considered elevated. In certain embodiments, an individual is selected to receive HDACi and antiviral according to the schedule described herein if they possess a serum creatinine level that exceeds 1.1 mg/dL for a woman or 1.3 mg/dL for a man. In certain embodiments, a patient can be selected to receive HDACi and antiviral according to the schedule described herein if they possess a blood urea nitrogen level in excess of the normal range. In certain embodiments, 20 milligrams per deciliter BUN exceeds the normal range.

The dosing schedules of HDACi and antivirals described herein can also be deployed prospectively to prevent certain side-effects in at risk individuals. In certain embodiments, a patient can be selected to receive HDACi and antiviral according to a schedule described herein if they possess a risk factor for compromised kidney function or thrombocytopenia. Risk factors for compromised kidney function comprise preexisting kidney disease, receipt of a kidney transplant, diabetes, high blood pressure, family history of kidney disease, advanced age, or African-American, Asian, Native American, or Hispanic ethnicity. Risk factors for thrombocytopenia comprise previous treatment with chemotherapy or radiation therapy, a history of anemia or thrombocytopenia.

In certain embodiments, the individual selected to be treated by the methods and schedules described herein is positive for the human immunodeficiency virus (HIV).

### Types of Viruses and Virally-Induced Cancers

The methods and compositions provided herein can be used to treat and/or prevent viral associated cancers. The virus causing the infection can be a member of the herpesvirus family, a human immunodeficiency virus, parvovirus, or coxsackie virus. A member of the herpesvirus family can be herpes simplex virus, herpes genitalis virus, varicella zoster virus, Epstein-Barr virus, human herpesvirus 6, human herpesvirus 8, or cytomegalovirus. The subject can have coronary artery condition associated with a cytomegalovirus or herpes simplex virus infection. The subject can have an autoimmune condition associated with Epstein-Barr virus infection. The subject can have a lymphoma or other cancer associated with Epstein-Barr virus infection. The subject can have a lymphoma or other cancer associated with human herpesvirus 8 infection. The subject can have an autoimmune condition associated with Herpes simplex virus infection. The subject can have a cancer associate with herpes simplex virus. The subject can have an autoimmune condition associated with cytomegalovirus infection. The subject can have a lymphoma or other cancer associated with cytomegalovirus infection.

The method described herein can be used to treat a solid tumor or cancer. In certain embodiments, the solid cancer or tumor is associated with Epstein-Barr virus or cytomegalovirus. In certain embodiments, the solid cancer is salivary gland cancer, nasopharyngeal carcinoma, head and neck cancer, gastric cancer, a colorectal cancer, breast cancer, glioblastoma, prostate cancer, renal cancer, pancreatic cancer, or lung cancer. In certain embodiments, the solid tumor or cancer is herein the solid tissue cancer is salivary gland cancer, nasopharyngeal carcinoma, head and neck cancer, gastric cancer, a colorectal cancer, or leiomyosarcoma. In certain embodiments, the solid tumor or cancer is salivary gland cancer. In certain embodiments, the solid tumor or cancer is nasopharyngeal carcinoma. In certain embodiments, the solid tumor or cancer is head and neck cancer. In certain embodiments, the solid tumor or cancer is Kaposi's sarcoma. In certain embodiments, the solid tumor or cancer is gastric cancer. In certain embodiments, the solid tumor or cancer is colorectal cancer. In certain embodiments, the solid tumor or caner is positive for Epstein-Barr virus. In certain embodiments, the solid tumor or caner is positive for cytomegalovirus.

The method described herein can be used to treat a hematologic tumor or cancer. In certain embodiments, the hematologic tumor or cancer comprises leukemia or a lymphoma. In certain embodiments, the leukemia or lymphoma is associated with Epstein-Barr virus or cytomegalovirus. In certain embodiments, the hematologic cancer is leukemia or a lymphoma. In certain embodiments, the leukemia or lymphoma is a B cell leukemia or lymphoma. In certain embodiments, the leukemia or lymphoma is a T cell leukemia or lymphoma. In certain embodiments, the leukemia or lymphoma is non-Hodgkin's lymphoma. In certain embodiments, the leukemia or lymphoma is Hodgkin's lymphoma. In certain embodiments, the leukemia or lymphoma is a cytomegalovirus virus positive leukemia or lymphoma. In certain embodiments, the leukemia or lymphoma is an Epstein-Barr virus positive leukemia or lymphoma.

### Formulations, Routes of Administration, and Effective Doses

Another aspect of the present invention relates to formulations, routes of administration and effective doses for pharmaceutical compositions comprising an agent or combination of agents. Such pharmaceutical compositions can be used to treat a virus-induced inflammatory condition as described above. A pharmaceutical composition can comprise a viral inducing agent. A pharmaceutical composition can comprise a viral inducing agent and one or more additional agents. A pharmaceutical composition can comprise an antiviral agent. A pharmaceutical composition can comprise an antiviral agent and one or more additional agents. A pharmaceutical composition can comprise a viral inducing agent and an antiviral agent. A pharmaceutical composition can comprise a viral inducing agent, an antiviral agent, and one or more additional agents.

The agents or their pharmaceutically acceptable salts can be provided alone or in combination with one or more other agents or with one or more other forms. For example, a formulation can comprise one or more agents in particular proportions, depending on the relative potencies of each agent and the intended indication. For example, in compositions for targeting two different targets and where potencies are similar, about a 1:1 ratio of agents can be used. The two forms can be formulated together, in the same dosage unit e.g. in one cream, suppository, tablet, capsule, enteric coated tablet or capsule, aerosol spray, or packet of powder to be dissolved in a beverage; or each form may be formulated in a separate unit, e.g., two creams, two suppositories, two tablets, two capsules, a tablet and a liquid for dissolving the tablet, two aerosol sprays, or a packet of powder and a liquid for dissolving the powder, etc.

A “pharmaceutically acceptable salt” can be a salt that retains the biological effectiveness and properties of one or more agents, and which are not biologically or otherwise undesirable. For example, a pharmaceutically acceptable salt does not interfere with the beneficial effect of a viral inducing agent or an antiviral agent.

Salts can include those of the inorganic ions, for example, sodium, potassium, calcium, magnesium ions, and the like. Salts can include salts with inorganic or organic acids, for example, hydrochloric acid, hydrobromic acid, phosphoric acid, nitric acid, sulfuric acid, methanesulfonic acid, p-toluenesulfonic acid, acetic acid, fumaric acid, succinic acid, lactic acid, mandelic acid, malic acid, citric acid, tartaric acid or maleic acid. If one or more agents contain a carboxy group or other acidic group, it can be converted into a pharmaceutically acceptable addition salt with inorganic or organic bases. Examples of suitable bases include sodium hydroxide, potassium hydroxide, ammonia, cyclohexylamine, dicyclohexyl-amine, ethanolamine, diethanolamine, triethanolamine, and the like.

A pharmaceutically acceptable ester or amide can be an ester or amide that retains biological effectiveness and properties of one or more agents, and which are not biologically or otherwise undesirable. For example, the ester or amide does not interfere with the beneficial effect of a viral inducing agent, an antiviral agent, or an additional agent. Esters can include, for example, ethyl, methyl, isobutyl, ethylene glycol, and the like. Amides include can include, for example, unsubstituted amides, alkyl amides, dialkyl amides, and the like.

One or more agents and/or combinations of agents can be administered with still other agents. The choice of agents that can be co-administered with the agents and/or combinations of agents can depend, at least in part, on the condition being treated. Agents of particular use in the formulations of the present invention include, for example, any agent having a therapeutic effect for a virus-induced inflammatory condition, including, e.g., drugs used to treat inflammatory conditions. For example, formulations of the instant invention can additionally contain one or more conventional anti-inflammatory drugs, such as an NSAID, e.g. ibuprofen, naproxen, acetominophen, ketoprofen, or aspirin. In some alternative embodiments, for the treatment of a virus-induced inflammatory condition can additionally contain one or more conventional influenza antiviral agents, such as amantadine, rimantadine, zanamivir, and oseltamivir. In treatments for retroviral infections, such as HIV, formulations of the instant invention may additionally contain one or more conventional antiviral drug, such as protease inhibitors (lopinavir/ritonavir {Kaletra™}, indinavir {Crixivan™}, ritonavir {Norvir™}, nelfinavir {Viracept™}, saquinavir hard gel capsules {Invirase™}, atazanavir {Reyataz™}, amprenavir {Agenerase™}, fosamprenavir {Telzir™}, tipranavir{Aptivus™}), reverse transcriptase inhibitors, including non-Nucleoside and Nucleoside/nucleotide inhibitors(AZT {zidovudine, Retrovir™},ddI {didanosine, Videx™}, 3TC {lamivudine, Epivir™}, d4T {stavudine, Zerit™}, abacavir {Ziagen™}, FTC {emtricitabine, Emtriva™}, tenofovir {Viread™}, efavirenz {Sustiva™} and nevirapine {Viramune™}), fusion inhibitors T20 {enfuvirtide, Fuzeon™}, integrase inhibitors (MK-0518 and GS-9137), and maturation inhibitors (PA-457 {Bevirimat™}). As another example, formulations can additionally contain one or more supplements, such as vitamin C, E or other anti-oxidants.

One or more agents (or pharmaceutically acceptable salts, esters or amides thereof) can be administered per se or in the form of a pharmaceutical composition wherein the one or more active agent(s) is in an admixture or mixture with one or more pharmaceutically acceptable carriers. A pharmaceutical composition, as used herein, can be any composition prepared for administration to a subject. Pharmaceutical compositions can be formulated in conventional manner using one or more physiologically acceptable carriers, comprising excipients, diluents, and/or auxiliaries, e.g., that facilitate processing of the active agents into preparations that can be administered. Proper formulation can depend at least in part upon the route of administration chosen. One or more agents, or pharmaceutically acceptable salts, esters, or amides thereof, can be delivered to a patient using a number of routes or modes of administration, including oral, buccal, topical, rectal, transdermal, transmucosal, subcutaneous, intravenous, and intramuscular applications, as well as by inhalation.

For oral administration, one or more agents can be formulated readily by combining the one or more active agents with pharmaceutically acceptable carriers well known in the art. Such carriers can enable the one or more agents to be formulated as tablets, including chewable tablets, pills, dragees, capsules, lozenges, hard candy, liquids, gels, syrups, slurries, powders, suspensions, elixirs, wafers, and the like, for oral ingestion by a patient to be treated. Such formulations can comprise pharmaceutically acceptable carriers including solid diluents or fillers, sterile aqueous media and various non-toxic organic solvents. Generally, the agents of the invention can be included at concentration levels ranging from about 0.5%, about 5%, about 10%, about 20%, or about 30% to about 50%, about 60%, about 70%, about 80% or about 90% by weight of the total composition of oral dosage forms, in an amount sufficient to provide a desired unit of dosage.

Aqueous suspensions for oral use can contain one or more agents with pharmaceutically acceptable excipients, such as a suspending agent (e.g., methyl cellulose), a wetting agent (e.g., lecithin, lysolecithin and/or a long-chain fatty alcohol), as well as coloring agents, preservatives, flavoring agents, and the like.

Oils or non-aqueous solvents can be required to bring one or more agents into solution, due to, for example, the presence of large lipophilic moieties. Alternatively, emulsions, suspensions, or other preparations, for example, liposomal preparations, can be used. With respect to liposomal preparations, any known methods for preparing liposomes for treatment of a condition can be used. See, for example, Bangham et al., J. Mol. Biol. 23: 238-252 (1965) and Szoka et al., Proc. Natl Acad. Sci. USA 75: 4194-4198 (1978), incorporated herein by reference. Ligands can also be attached to the liposomes to direct these compositions to particular sites of action. One or more agents can also be integrated into foodstuffs, e.g., cream cheese, butter, salad dressing, or ice cream to facilitate solubilization, administration, and/or compliance in certain patient populations.

Pharmaceutical preparations for oral use can be obtained as a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; flavoring elements, cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinyl pyrrolidone (PVP). Disintegrating agents can be added, for example, the cross-linked polyvinyl pyrrolidone, agar, or alginic acid or a salt thereof such as sodium alginate. One or more agents can also be formulated as a sustained release preparation.

Dragee cores can be provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of one or more active agents.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active agents can be dissolved or suspended in suitable liquids, such as fatty oils, liquid paraffin, or liquid polyethylene glycols. In addition, stabilizers can be added. All formulations for oral administration can be in dosages suitable for administration.

For injection, one or more agents can be formulated in aqueous solutions, including but not limited to physiologically compatible buffers such as Hank's solution, Ringer's solution, or physiological saline buffer. Such compositions can also include one or more excipients, for example, preservatives, solubilizers, fillers, lubricants, stabilizers, albumin, and the like. Methods of formulation are known in the art, for example, as disclosed in Remington's Pharmaceutical Sciences, latest edition, Mack Publishing Co., Easton P.

One or more agents can also be formulated as a depot preparation. Such long acting formulations can be administered by implantation or transcutaneous delivery (for example subcutaneously or intramuscularly), intramuscular injection or use of a transdermal patch. Thus, for example, one or more agents can be formulated with suitable polymeric or hydrophobic materials (for example as an emulsion in an acceptable oil) or ion exchange resins, or as sparingly soluble derivatives, for example, as a sparingly soluble salt.

Pharmaceutical compositions comprising one or more agents can exert local and regional effects when administered topically or injected at or near particular sites of infection. Direct topical application, e.g., of a viscous liquid, gel, jelly, cream, lotion, ointment, suppository, foam, or aerosol spray, can be used for local administration, to produce, for example local and/or regional effects. Pharmaceutically appropriate vehicles for such formulation include, for example, lower aliphatic alcohols, polyglycols (e.g., glycerol or polyethylene glycol), esters of fatty acids, oils, fats, silicones, and the like. Such preparations may also include preservatives (e.g., p-hydroxybenzoic acid esters) and/or antioxidants (e.g., ascorbic acid and tocopherol). See also Dermatological Formulations: Percutaneous absorption, Barry (Ed.), Marcel Dekker Incl, 1983. In some embodiments, local/topical formulations comprising a viral inducing agent and or antiviral agent are used to treat epidermal or mucosal viral-induced inflammatory condition.

Pharmaceutical compositions can contain a cosmetically or dermatologically acceptable carrier. Such carriers can be compatible with skin, nails, mucous membranes, tissues and/or hair, and can include any conventionally used cosmetic or dermatological carrier meeting these requirements. Such carriers can be readily selected by one of ordinary skill in the art. In formulating skin ointments, an agent or combination of agents can be formulated in an oleaginous hydrocarbon base, an anhydrous absorption base, a water-in-oil absorption base, an oil-in-water water-removable base and/or a water-soluble base.

The compositions according to the present invention can be in any form suitable for topical application, including aqueous, aqueous-alcoholic or oily solutions, lotion or serum dispersions, aqueous, anhydrous or oily gels, emulsions obtained by dispersion of a fatty phase in an aqueous phase (O/W or oil in water) or, conversely, (W/O or water in oil), microemulsions or alternatively microcapsules, microparticles or lipid vesicle dispersions of ionic and/or nonionic type. These compositions can be prepared according to conventional methods. The amounts of the various constituents of the compositions according to the invention can be those conventionally used in the art. These compositions constitute protection, treatment or care creams, milks, lotions, gels or foams for the face, for the hands, for the body and/or for the mucous membranes, or for cleansing the skin. The compositions can also consist of solid preparations constituting soaps or cleansing bars.

A pharmaceutical composition can also contain adjuvants common to the cosmetic and dermatological fields, for example, hydrophilic or lipophilic gelling agents, hydrophilic or lipophilic active agents, preserving agents, antioxidants, solvents, fragrances, fillers, sunscreens, odor-absorbers and dyestuffs. The amounts of these various adjuvants can be those conventionally used in the fields considered and, for example, are from about 0.01% to about 20% of the total weight of the composition. Depending on their nature, these adjuvants can be introduced into the fatty phase, into the aqueous phase and/or into the lipid vesicles.

Ocular viral infections can be effectively treated with ophthalmic solutions, suspensions, ointments or inserts comprising an agent or combination of agents of the present invention.

In some embodiments, viral infections of the ear can be effectively treated with otic solutions, suspensions, ointments or inserts comprising an agent or combination of agents of the present invention.

One or more agents can be delivered in soluble rather than suspension form, which can allow for more rapid and quantitative absorption to the sites of action. In general, formulations such as jellies, creams, lotions, suppositories and ointments can provide an area with more extended exposure to the agents of the present invention, while formulations in solution, e.g., sprays, provide more immediate, short-term exposure.

Relating to topical/local application, a pharmaceutical composition can include one or more penetration enhancers. For example, the formulations can comprise suitable solid or gel phase carriers or excipients that increase penetration or help delivery of agents or combinations of agents of the invention across a permeability barrier, e.g., the skin. Many of these penetration-enhancing compounds are known in the art of topical formulation, and include, e.g., water, alcohols (e.g., terpenes like methanol, ethanol, 2-propanol), sulfoxides (e.g., dimethyl sulfoxide, decylmethyl sulfoxide, tetradecylmethyl sulfoxide), pyrrolidones (e.g., 2-pyrrolidone, N-methyl-2-pyrrolidone, N-(2-hydroxyethyl)pyrrolidone), laurocapram, acetone, dimethylacetamide, dimethylformamide, tetrahydrofurfuryl alcohol, L-α-amino acids, anionic, cationic, amphoteric or nonionic surfactants (e.g., isopropyl myristate and sodium lauryl sulfate), fatty acids, fatty alcohols (e.g., oleic acid), amines, amides, clofibric acid amides, hexamethylene lauramide, proteolytic enzymes, α-bisabolol, d-limonene, urea and N,N-diethyl-m-toluamide, and the like. Additional examples include humectants (e.g., urea), glycols (e.g., propylene glycol and polyethylene glycol), glycerol monolaurate, alkanes, alkanols, ORGELASE, calcium carbonate, calcium phosphate, various sugars, starches, cellulose derivatives, gelatin, and/or other polymers. A pharmaceutical composition can include one or more such penetration enhancers.

A pharmaceutical composition for local/topical application can include one or more antimicrobial preservatives, for example, quaternary ammonium compounds, organic mercurials, p-hydroxy benzoates, aromatic alcohols, chlorobutanol, and the like.

Gastrointestinal viral infections can be effectively treated with orally- or rectally delivered solutions, suspensions, ointments, enemas and/or suppositories comprising an agent or combination of agents of the present invention.

Respiratory viral infections can be effectively treated with aerosol solutions, suspensions or dry powders comprising an agent or combination of agents of the present invention. Administration by inhalation is particularly useful in treating viral infections of the lung, such as influenza. The aerosol can be administered through the respiratory system or nasal passages. For example, one skilled in the art will recognize that a composition of the present invention can be suspended or dissolved in an appropriate carrier, e.g., a pharmaceutically acceptable propellant, and administered directly into the lungs using a nasal spray or inhalant. For example, an aerosol formulation comprising a viral inducing agent and/or antiviral agent can be dissolved, suspended or emulsified in a propellant or a mixture of solvent and propellant, e.g., for administration as a nasal spray or inhalant. Aerosol formulations may contain any acceptable propellant under pressure, such as a cosmetically or dermatologically or pharmaceutically acceptable propellant, as conventionally used in the art.

An aerosol formulation for nasal administration is generally an aqueous solution designed to be administered to the nasal passages in drops or sprays. Nasal solutions can be similar to nasal secretions in that they are generally isotonic and slightly buffered to maintain a pH of about 5.5 to about 6.5, although pH values outside of this range can additionally be used. Antimicrobial agents or preservatives can also be included in the formulation.

An aerosol formulation for inhalations and inhalants can be designed so that an agent or combination of agents can be carried into the respiratory tree of the subject when administered by the nasal or oral respiratory route. Inhalation solutions can be administered, for example, by a nebulizer. Inhalations or insufflations, comprising finely powdered or liquid drugs, can be delivered to the respiratory system as a pharmaceutical aerosol of a solution or suspension of the agent or combination of agents in a propellant, e.g., to aid in disbursement. Propellants can be liquefied gases, including halocarbons, for example, fluorocarbons such as fluorinated chlorinated hydrocarbons, hydrochlorofluorocarbons, and hydrochlorocarbons, as well as hydrocarbons and hydrocarbon ethers.

Halocarbon propellants can include fluorocarbon propellants in which all hydrogens are replaced with fluorine, chlorofluorocarbon propellants in which all hydrogens are replaced with chlorine and at least one fluorine, hydrogen-containing fluorocarbon propellants, and hydrogen-containing chlorofluorocarbon propellants. Halocarbon propellants are described in Johnson, U.S. Pat. No. 5,376,359, issued Dec. 27, 1994; Byron et al., U.S. Pat. No. 5,190,029, issued Mar. 2, 1993; and Purewal et al., U.S. Pat. No. 5,776,434, issued Jul. 7, 1998. Hydrocarbon propellants useful in the invention include, for example, propane, isobutane, n-butane, pentane, isopentane and neopentane. A blend of hydrocarbons can also be used as a propellant. Ether propellants include, for example, dimethyl ether as well as the ethers. An aerosol formulation of the invention can also comprise more than one propellant. For example, an aerosol formulation can comprise more than one propellant from the same class, such as two or more fluorocarbons; or more than one, more than two, more than three propellants from different classes, such as a fluorohydrocarbon and a hydrocarbon. Pharmaceutical compositions of the present invention can also be dispensed with a compressed gas, e.g., an inert gas such as carbon dioxide, nitrous oxide or nitrogen.

Aerosol formulations can also include other components, for example, ethanol, isopropanol, propylene glycol, as well as surfactants or other components such as oils and detergents. These components can serve to stabilize the formulation and/or lubricate valve components.

The aerosol formulation can be packaged under pressure and can be formulated as an aerosol using solutions, suspensions, emulsions, powders and semisolid preparations. For example, a solution aerosol formulation can comprise a solution of an agent, such as a viral inducing agent and/or antiviral agent in (substantially) pure propellant or as a mixture of propellant and solvent. The solvent can be used to dissolve the agent and/or retard the evaporation of the propellant. Solvents useful in the invention include, for example, water, ethanol and glycols. Any combination of suitable solvents can be used, optionally combined with preservatives, antioxidants, and/or other aerosol components.

An aerosol formulation can also be a dispersion or suspension. A suspension aerosol formulation may comprise a suspension of an agent or combination of agents of the instant invention, e.g., a viral inducing agent and/or antiviral agent, and a dispersing agent. Dispersing agents useful in the invention include, for example, sorbitan trioleate, oleyl alcohol, oleic acid, lecithin and corn oil. A suspension aerosol formulation can also include lubricants, preservatives, antioxidant, and/or other aerosol components.

An aerosol formulation can be formulated as an emulsion. An emulsion aerosol formulation can include, for example, an alcohol such as ethanol, a surfactant, water and a propellant, as well as an agent or combination of agents, e.g., a viral inducing agent and/or an antiviral agent. The surfactant used can be nonionic, anionic or cationic. One example of an emulsion aerosol formulation comprises, for example, ethanol, surfactant, water and propellant. Another example of an emulsion aerosol formulation comprises, for example, vegetable oil, glyceryl monostearate and propane.

Pharmaceutical compositions suitable for use in the present invention can include compositions wherein the active ingredients are present in an effective amount, i.e., in an amount effective to achieve therapeutic and/or prophylactic benefit in a host with at least one virus-induced inflammatory condition. The actual amount effective for a particular application will depend on the condition or conditions being treated, the condition of the subject, the formulation, and the route of administration, as well as other factors known to those of skill in the art. Determination of an effective amount of a viral inducing agent and/or antiviral agent is well within the capabilities of those skilled in the art, in light of the disclosure herein, and can be determined using routine optimization techniques.

An effective amount for use in humans can be determined from animal models. For example, a dose for humans can be formulated to achieve circulating, liver, topical and/or gastrointestinal concentrations that have been found to be effective in animals. One skilled in the art can determine the effective amount for human use, especially in light of the animal model experimental data described herein. Based on animal data, and other types of similar data, those skilled in the art can determine an effective amount of a composition appropriate for humans.

An effective amount when referring to an agent or combination of agents of the invention can generally mean the dose ranges, modes of administration, formulations, etc., that have been recommended or approved by any of the various regulatory or advisory organizations in the medical or pharmaceutical arts (e.g., FDA, AMA) or by the manufacturer or supplier.

Further, appropriate doses for a viral inducing agent and/or antiviral agent can be determined based on in vitro experimental results.

A person of skill in the art would be able to monitor in a patient the effect of administration of a particular agent. For example, HIV or EBV viral load levels can be determined by techniques standard in the art, such as measuring CD4 cell counts, and/or viral levels as detected by PCR. Other techniques would be apparent to one of skill in the art.

This disclosure provides for a kit, the kits can comprise one or more containers, the kit can comprise any combination of HDAC inhibitors, antivirals or additional agents mentioned in this disclosure in suitable packaging. The kit may contain instructions for use. The HDAC inhibitor or antiviral can be present in any concentration disclosed herein, can be packaged for administration by any route disclosed herein, or in any formulation disclosed herein. In some embodiments, the HDAC and antiviral agent are packaged together, in a suitable package or container, in a kit. The kit may be for convenient administration or dosing, and management thereof. In some further embodiments, the HDAC inhibitor and antiviral are formulated together as a pharmaceutical composition in a single dose. In some alternative embodiments, the HDAC inhibitor and antiviral are formulated as separate pharmaceutical compositions. In some embodiments, the pharmaceutical composition of the HDAC inhibitor is packaged for once a week, twice a week, thrice a week, four times a week or more, once a month, twice a month, thrice a month, four times a month or more dosing; and the pharmaceutical composition of the antiviral is packaged for daily, twice daily, thrice daily, four times a day or more dosing. In some embodiments, the antiviral is administered or taken without the HDAC inhibitor. In some embodiments, the treatment course of the HDAC inhibitor and antiviral can be as follows: the HDAC inhibitor and the antiviral are taken or administered together in the same pharmaceutical composition on any of the first, second, third, fourth, fifth or more days of treatment; and the antiviral is taken or administered by itself on any of days 2, 3, 4, 5, 6, 7, 8, 9, 10, 11, 12, 13 or more. In some further embodiments, the treatment course can be as follows: the HDAC inhibitor and antiviral are taken or administered separately in different pharmaceutical composition on any of the first, second, third, fourth, fifth or more days of treatment, either at the same time or temporally separated by 1, 2, 3, 4, 5, 6, 7, 8, 9, 10, 11, 12, or more hours; and the antiviral is taken or administered by itself on any of days 2, 3, 4, 5, 6, 7, 8, 9, 10, 11, 12, 13 or more. In some embodiments, the HDAC inhibitor packaged in the kit is chidamide in other embodiments, it is 4SC-202. In some embodiments, the antiviral is ganciclovir, in other embodiments, it is valganciclovir. In some embodiments, the treatment course is repeated for 1, 2, 3, 4, 5, 6, 7, 8, 9, 10, 11, 12, 13, 14 or more iterations.

The kits of this invention are in suitable packaging. Suitable packaging includes, but is not limited to, vials, bottles, jars, flexible packaging (e.g., sealed Mylar, blister packs, plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is an HDAC inhibitor. The HDAC inhibitor can be 4SC-202 or chidamide. The container may further comprise a second pharmaceutically active agent. This second pharmaceutically active agent can be an antiviral. The antiviral can be ganciclovir or valganciclovir.

### EXAMPLES

### Example 1—Phase One Clinical Trial Combining HDACi and Antiviral Treatment

Nanatinostat (Nstat) (VRx-3996 formerly known as CHR-3996) is a Class 1-selective, oral, hydroxamate histone deacetylase (HDAC) inhibitor active against HDAC 1-3 but not HDAC 6. Single-agent Nstat has previously been evaluated in Phase 1 study in patients with solid tumors.

Ontract (NCT03397706) is a Phase 1b/2, open-label, dose-escalation (3+3 design) study followed by an expansion stage at the recommended Phase 2 dose (RP2D) of p.o. N+VG in patients with EBV-associated lymphomas. Dose schedules tested are shown in Table 1 below.

TABLE 1
Dose schedules and amounts

| | |
|---|---|
| | Phase 1b Escalation |
| Cohort | Dose/Schedule |

| | | |
|---|---|---|
| 1 | N 10 mg BID, daily | VG 900 mg BID |
| 2a | N 5 mg BID, daily | VG 450 mg BID |
| 2b | N 10 mg QD, daily | VG 450 mg BID |
| 2c | N 10 mg QD, daily | VG 900 mg QD |
| 3 | N 20 mg QD × 4 days per week | VG 900 mg QD |
| | (3 days off); weekly × 4 | |

### Primary Objectives

Phase 1b: Determine the safety, tolerability, and R2PD of N+VG
Phase 2: Evaluate the safety and tolerability of the R2PD and assess the objective response rate (ORR) by Recommendations for Initial Evaluation, Staging, and Response Assessment of Hodgkin and Non-Hodgkin Lymphoma: The Lugano Classification⁷ (Lugano) and RECIL 2017⁸ by central review

### Secondary Objectives

Phase 1b/2: Evaluate pharmacokinetics (PK) of N and PK of ganciclovir
Phase 2: Evaluate time to tumor response, duration of response, time to tumor progression, and progression-free survival

### Exploratory Outcomes

Phase 1b/2: Evaluate changes in viral loads (CMV, EBV, HHV-6, HHV-8, HIV) as appropriate, EBV latency/lytic profile and genome methylation status, changes in histone H3 acetylation (PBMCs)

### Key Eligibility Criteria

Relapsed/refractory, pathologically confirmed EBV+ lymphoma (EBER-ISH) or lymphoproliferative disease regardless of histologic subtype, including:
EBV-associated post-transplant lymphoproliferative disease (PTLD) after allogeneic hematopoietic cell transplant (alloHCT) or solid organ transplant (SOT)
EBV-associated lymphoproliferative disorders (LPD) and malignancies associated with acquired immunodeficiency, including HIV+ infections
EBV-associated lymphomas and LPDs not associated with immunodeficiency
Presence of measurable disease
Age ≥18 years; ECOG performance status of 0 to 2; adequate hematologic, renal, and hepatic function

The demographics of individuals enrolled in the trial are shown in Table 2 and the dosing by Cohort and intensity is shown in Table 3 below.

TABLE 2
Demographics and Lymphoma Characteristics

| Patients Enrolled (n) | 25 | |
|---|---|---|
| Male/Female | 18/7 | |
| Median Age, (Range), yrs | 58 | (19-84) |
| ECOG PS, 0, 1, or 2 | 8, 15, 2 | |
| Median Prior Therapies, n (Range) | 2 | (1-9) |
| Irradiation | 6 | |
| Transplant, n (%) [Auto, Allo, | 9 | [7, 1, 1] |
| Both] | | |
| HDACi | 3 (2 = Romidepsin, 1 Belinosat) | |
| Median Day-1 Platelets, 10⁹/L, | 174 | (64-327) |
| (Range) | | |

Lymphoma type

| B cell (12 total) | T-cell (8 total) | |
|---|---|---|
| DLBCL (5) | Extranodal NK/T Cell Lymphoma (3) | |
| Plasmablastic lymphoma (2) | Cutaneous T-cell lymphoma (1) | |
| Burkitt's (1) | Anaplastic T cell (3) | |
| Lymphoplasmacytic LPD (1) | Peripheral T-cell lymphoma | |
| Post-transplant lymphoproliferative | not otherwise specified (1) | |
| disorders (1) | | |
| Other (2) | | |
| Hodgkin's (5) | | |
| HIV+ | 4 | (20%) |

TABLE 3
Dosing by Cohort and Intensity

| Cohort | 1 | 2a | 2b | 2c |
|---|---|---|---|---|
| Total Daily Dose | N 20 mg | N 10 mg | | |
| | VG 1800 mg | VG 900 mg | | |
| Schedule | 10 BID | 5 BID | 10 QD | 10 QD |
| | 900 BID | 450 BID | 450 BID | 900 QD |
| Patients (n) | 7 | 5 | 4 | 4 |
| N 28-Day | 13.8 mg | 8.8 mg | 10 mg | 7.8 mg |
| Median Daily Dose | (69%) | (88%) | (100%) | (78%) |
| Intensity, (%) | [9.3-20] | [0-10] | [1-10] | [0-10] |
| [range] | | | | |

*VG starting dose adjusted for renal function per prescribing guidelines

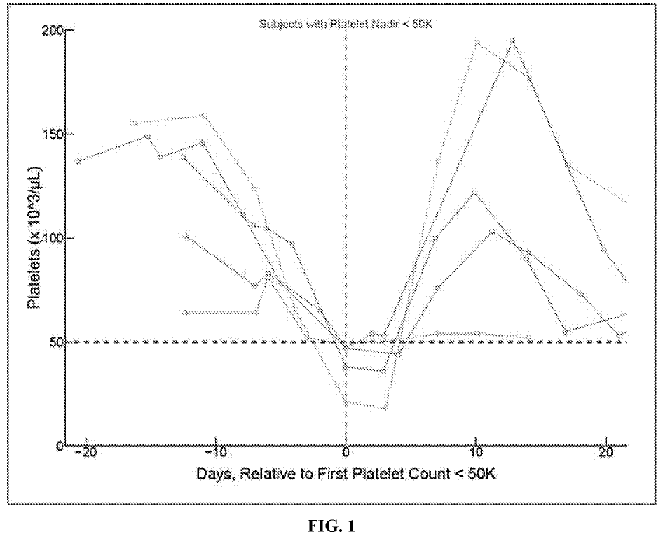

Cohort 1 (10 mg, BID) exceeded the maximum tolerated doses based on 4 different hematologic dose limiting toxicities in 2 patients. No dose limiting toxicities and fewer dose holds were observed in cohort 2 (5 mg BID or 10 mg QD). Thrombocytopenia was the most common dose limiting toxicity as shown in Table 4. Platelet nadirs rapidly recovered after 3 to 5 days of dose hold. See FIG. 1. This indicated that treatment according to a schedule where patients were treated with HDACi for 1, 2, 3, 4, or 5 days; and not treated for 6, 5, 4, 3, or 2 days might limit hematologic side effects like thrombocytopenia.

TABLE 4

| | | | |
|---|---|---|---|
| | | | Cohort 3 (N = 5) + Phase |
| | Cohort 1 (N = 7) | Cohort 2abc (N = 13) | 2 (N = 8) at the RP2D |
| | [Nstat 20 mg, | [Nstat 10 mg, | [Nstat 20 mg (4/7 d), |
| Adverse Event | VGCV 1800 mg] | VGCV 900 mg] | VGCV 900 mg] N (%) |

Hematologic

| | | | |
|---|---|---|---|
| Thrombocytopenia | 3 (43%) | 2 (15%) | 0 |
| Neutropenia | 2 (28%) | 2 (15%) | 1 (8%) |
| Anemia | 1 (14%) | 1 (8%) | 1 (8%) |

Non-Hematologic

| | | | |
|---|---|---|---|
| Creatinine | 0 | 0 | 0 |
| Elevation | | | |
| Fatigue | 1 (14%) | 0 | 0 |

Nstat = nanatinostat; VGCV = valganciclovir; 4/7 d = 4 days of treatment in 7-day schedule

Overall, Objective responses were observed in all dose cohorts and across B- and T-cell lymphomas. See Table 5.Evidence of antitumor activity was observed by PET in 9 of 12 patients evaluable by PET. Major responses have been observed at the first scan at week 8. Interestingly, pseudo-progression was observed in two patients at approximately month 4 were followed by major responses (CR, PR), and may indicate immune surveillance response. In two patients, progression in the skin was observed while maintaining a CR or PR systemic response. One patient who had skin clearing 2 weeks after therapy discontinued treatment and has remained disease free. In HIV+ patients, 2 of 2 evaluable have progressed

TABLE 5

| Best | All | HIV-negative | | | | |
|---|---|---|---|---|---|---|
| Response | (n = 18) | All | B Cell | T cell | NK cell | Hodgkin |
| CR | 5 | 5 | 2 | 1 | 1 | 1 |
| PR | 5 | 5 | 2 | 2 | 2 | 0 |
| SD | 4 | 4 | 1 | 0 | 0 | 3 |
| PD | 4 | 1 | 1 | 0 | 0 | 0 |
| Total | 18 | 15 | 5 | 3 | 3 | 4 |

CR = complete response; PR = partial response; SD = stable disease; PD-progressive disease

### Example 2—Nstat is More Potent than Other HDACi and Treatment Results in Long Term Histone Acetylation

Nanatinostat has T_{1/2} of approximately 2 hours as shown in Table 6, in experiments conducted in humans. Patients enrolled in study were administered oral nanatinostat and valganciclovir at predefined doses. Blood samples for pharmacokinetic studies were drawn on cycle 1 day 1 at 30 minutes, 1, 2, 4 6 and 24 hours after the first dose of nanatinostat. Serum concentration of nanatinostat were assayed at Inotiv, Inc. and PK parameters, including terminal half-life calculated using Phoenix WinNonlin v. 8.1 software.

TABLE 6
Geometric Mean (% CV) Single Dose Pharmacokinetics of VRx-3996 after Different Treatments

| Treatment | A | | | B | | | C | | |
|---|---|---|---|---|---|---|---|---|---|
| Parameter | N | Mean | % CV | N | Mean | % CV | N | Mean | % CV |
| Tₘₐₓ (h)* | 4 | 3.00 (1.00-6.00) | | 9 | 2.00 (1.00-6.00) | | 6 | 1.00 (0.500-6.00) | |
| Cₘₐₓ (ng/ml) | 4 | 18.5 | 230 | 9 | 62.6 | 138 | 6 | 78.9 | 46.3 |
| AUCₗₐₛₜ (ng-h/mL) | 4 | 40.5 | 204 | 9 | 165 | 128 | 6 | 227 | 37.8 |
| AUC (ng-h/mL) | 1 | 117 | NC | 1 | 317 | NC | 4 | 322 | 12.9 |
| Half-Life (h) | 1 | 2.18 | NC | 1 | 1.17 | NC | 4 | 1.67 | 21.1 |

A = 5 mg VRx-3996 p.o. and 450 mg Valganciclovir p.o.
B = 10 mg VRx-3996 p.o. and 450 mg Valganciclovir p.o.
C = 10 mg VRx-3996 p.o. and 900 mg Valganciclovir p.o.
*Median (range)

The IC₅₀ of HDAC inhibition for nanatinostat, romidepsin (FK228), entinostat (MS-275), suberanilohydroxamic acid (SAHA or vorinostat), or trichostatin A (TSA) was determined in an in vitro histone acetylation assay. Briefly, all of the compounds are dissolved in DMSO. A series of dilutions of the compounds were prepared with 10% DMSO in HDAC assay buffer and 5 μl of the dilution was added to a 50 μl reaction so that the final concentration of DMSO is 1% in all of reactions. The compounds were pre-incubated in duplicate at RT for 1 hour in a mixture containing HDAC assay buffer, 5 μg BSA, HDAC enzyme (see Table 7) and a the particular HDACi. After 1 hour, the enzymatic reactions were initiated by the addition of HDAC substrate (BPS Bioscience) to a final concentration of 10 μM or 2 μM. The enzymatic reaction proceeded for 30 minutes at 37° C. After enzymatic reactions, 50 μl of 2×HDAC Developer was added to each well for the HDAC enzymes and the plate was incubated at room temperature for an additional 15 minutes. Fluorescence intensity was measured at an excitation of 360 nm and an emission of 460 nm using a Tecan Infinite M1000 microplate reader.

HDAC activity assays were performed in duplicates at each concentration. The fluorescent intensity data were analyzed using the computer software, Graphpad Prism. In the absence of the compound, the fluorescent intensity (Fₜ) in each data set was defined as 100% activity. In the absence of HDAC, the fluorescent intensity (F_{b}) in each data set was defined as 0% activity. The percent activity in the presence of each compound was calculated according to the following equation: % activity=(F−F_{b})/(Fₜ−F_{b}), where F=the fluorescent intensity in the presence of the compound.

The values of % activity versus a series of compound concentrations were then plotted using non-linear regression analysis of Sigmoidal dose-response curve generated with the equation Y=B+(T−B)/1+10^{((Log C50-X)×Hill Slope)}, where Y=percent activity, B=minimum percent activity, T=maximum percent activity, X=logarithm of compound and Hill Slope=slope factor or Hill coefficient. The IC₅₀ value was determined by the concentration causing a half-maximal percent activity.

TABLE 7

| | Enzyme Used | |
|---|---|---|
| Assay | (ng)/Reaction | Substrate |
| HDAC1 | 7.2 | 10 μM HDAC Substrate 3 |
| HDAC2 | 7.5 | 10 μM HDAC Substrate 3 |
| HDAC3/NCOR2 | 3.4 | 10 μM HDAC Substrate 3 |
| HDAC4 | 0.3 | 2 μM HDAC Substrate Class 2a |
| HDAC5 | 40 | 2 μM HDAC Substrate Class 2a |
| HDAC6 | 10 | 10 μM HDAC Substrate 3 |
| HDAC7 | 1.6 | 2 μM HDAC Substrate Class 2a |
| HDAC8 | 25 | 2 μM HDAC Substrate Class 2a |
| HDAC9 | 4.3 | 2 μM HDAC Substrate Class 2a |
| HDAC11 | 60 | 2 μM HDAC Substrate Class 2a |

Results for these experiments are shown in Table 8. Overall, in comparison to other HIDACi, Nstat shows high potency inhibition of HDAC1, HDAC2, HDAC4, HDAC5, HDAC8, and HDAC9 when compared to all other HIDACi tested. Overall the results support a rationale to dose an HIDACi with an anti-viral, wherein the HDACi is dosed at a level that is below a maximum tolerated dose.

TABLE 8

| | IC₅₀ (μM) or Percentage Inhibition | | | | |
|---|---|---|---|---|---|
| Enzymes | Nstat | FK228 | MS-275 | SAHA | TSA |
| HDAC1 | 0.0036 | 1.2 | 0.19 | 0.087 | — |
| HDAC2 | 0.012 | 8.4 | 0.31 | 0.099 | — |
| HDAC3/NCOR2 | 0.012 | 4.3 | 0.28 | 0.078 | — |
| HDAC4 | 1 | >100 | >100 | >100 | 4.5 |
| | | NI* @ | NI @ | 34% @ | |
| | | 100 μM | 100 μM | 100 μM | |
| HDAC5 | 0.39 | >100 | >100 | ~48 | 3.4 |
| | | 17% @ | NI @ | | |
| | | 100 μM | 100 μM | | |
| HDAC6 | 0.45 | 0.74 | ~66 | 0.011 | — |
| HDAC7 | 1.6 | >100 | >100 | >100 | 1.4 |
| | | NI @ | NI @ | 27% @ | |
| | | 100 μM | 100 μM | 100 μM | |
| HDAC8 | 0.86 | ~63 | >100 | ~46 | 1.6 |
| | | | NI @ | | |
| | | | 100 μM | | |
| HDAC9 | 0.44 | >100 | >100 | >100 | 4.1 |
| | | NI @ | NI @ | 37% @ | |
| | | 100 μM | 100 μM | 100 μM | |
| HDAC11 | 2.8 | >100 | >100 | ~49 | 2.1 |
| | | NI @ | NI @ | | |
| | | 100 μM | 100 μM | | |

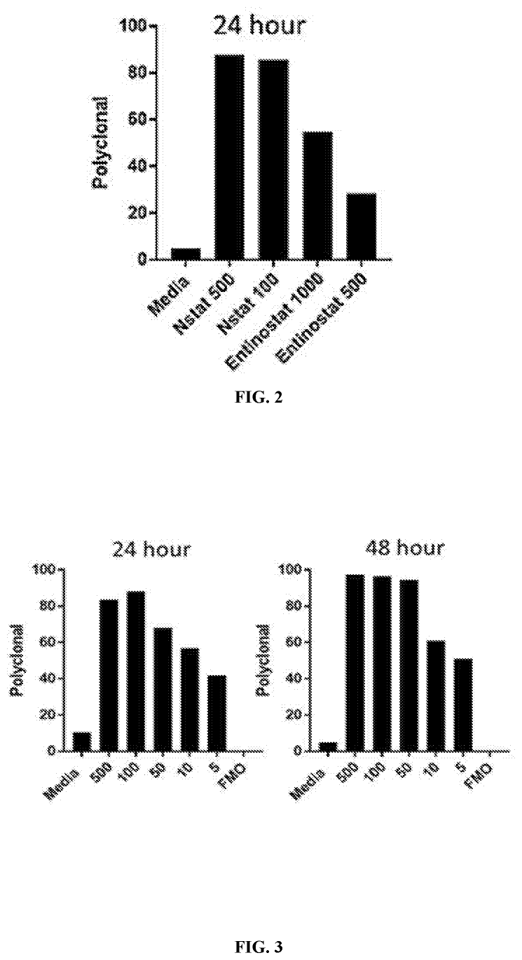

To determine if it was feasible to implement a dose hold from a pharmacokinetic standpoint the peripheral blood mononuclear cells were isolated from healthy volunteers and treated with either nanatinostat or entinostat (an HDACi with an elimination of half-life of approximately 36 hours). Since H3 acetylation is a pharmacodynamic biomarker of Nstat activity, acetylation of H3 was examined in treated cells. As shown in FIG. 2, Nstat was much more potent than entinostat in inducing elevated levels of Histone 3 acetylation, even at a dose of 100 nM. As shown in FIG. 3, this effect on H3 acetylation was long-lasting even at doses down to 10 nM.

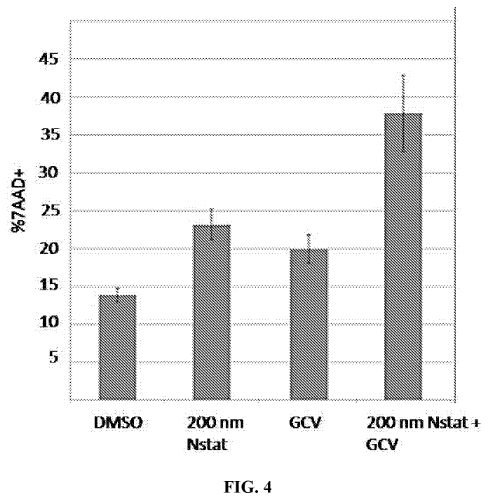

Nanatinostat increases cell cytotoxicity in an EBV infected cell line when combined with ganciclovir. This effect is seen even after Nstat removal as shown in FIG. 4. P3HR1 Burkitt's lymphoma cells were incubated with DMSO (solvent control) or Nstat for 3 days. After that time cell cultures were washed re-fed with ganciclovir (GCV) for an additional 3 days. All test conditions were run in triplicate. Cell death was measured by 7AAD staining.

Overall, these data and experiments provide rational that an HDACi, can be does at a level below the maximum tolerated dose to control adverse reactions while maintaining therapeutic efficiency.

### Example 3—Nstat and Valganciclovir Show Efficacy in Non-Hodgkin's Lymphoma

Background: EBV-positive (EBV+) lymphomas including Hodgkin, B and T cell lymphomas, are generally associated with poor clinical outcomes, particularly for patients (pts) who have relapsed or are refractory (R/R) to standard therapies. There are currently no approved therapies for EBV+ lymphomas and with the exception of adoptive T-cell therapies, no EBV-targeted antilymphoma therapeutics are in development. EBV is detectable in cancer cells by in situ hybridization for EBV-encoded RNAs (EBER-ISH). Nstat (VRx-3996), a Class I-selective oral hydroxamate histone deacetylase (HDAC) inhibitor active against HDAC1-3, induces the expression of EBV protein kinases which activate the anti-viral nucleoside analogue VGCV via mono-phosphorylation. This leads to inhibition of both viral and cellular DNA synthesis in EBV+tumor cells and potentially in surrounding EBV-tumor cells as well (bystander effect), causing apoptosis. This trial is the first to explore the safety and clinical activity of this targeted approach using oral Nstat in combination with oral VGCV in pts with R/R EBV+ lymphomas. Here we present an update of safety and efficacy for all enrolled patients, plus the preliminary safety of the recommended phase 2 doses (RP2D) of Nstat and VGCV (NCT03397706).

Methods: Pts with biopsy-proven EBV+ lymphomas (by EBER-ISH; any positive tumor cell) that had failed >1 prior systemic therapy and lacked treatment options by investigator's judgment were eligible for enrollment. Phase 1b used a 3×3 design to determine the RP2D of Nstat+VGCV. Phase 2 pts received the RP2D (Nstat 20 mg days (d) 1-4/7+VGCV 900 mg orally daily in 28 d cycles) until PD or withdrawal. Primary endpoints were safety/RP2D selection (phase 1b) and ORR (phase 2); secondary endpoints were pharmacokinetics, duration of response (DoR), time to response (TTR), progression free survival (PFS) and overall survival (OS). Response assessments began after Cycle 2 using Lugano 2014 response criteria.

Results: As of 5 Jul. 2020, 43 pts have enrolled (phase 1b: 25; phase 2: 18). Lymphoma subtypes were diffuse large B cell (DLBCL) (6), extranodal NK/T-cell (ENKTL) (6), peripheral T cell, NOS (PTCL-NOS) (3), angioimmunoblastic (AITL) (4), cutaneous T cell (CTCL) (1), Hodgkin (HL) (8), other B cell (2), and immunodeficiency-associated lymphoproliferative disorders (IA-LPD) (13), including post-transplant lymphoproliferative disorder (PTLD) (4),

HIV-associated (5), and other [4: systemic lupus erythematosus (SLE) (2), common variable immunodeficiency/primary immunodeficiency (2)]. Pts had a median of 2 prior therapies (range 1-11); 77% with ≥2 prior therapies, 86% were refractory to their most recent previous therapy and 77% had exhausted standard therapies in the judgment of the investigator. EBER positivity ranged from <1 to 80% in 18 pre-study tumor biopsies with central lab review. Most treatment related AEs (TRAEs) were mild or moderate, most commonly thrombocytopenia (33%), nausea(29%), neutropenia (26%) and fatigue (24%). At the RP2D, 23 pts were evaluable for safety. The most frequent G3/4 TRAEs (in ≥5% of pts) were neutropenia (14%), anemia (9%), and nausea (9%). For all evaluable pts (n=34), the ORR was 44% (15/34), with 8 (24%) complete responses (CR). The median TTR was 53 d (range 44-161 d). Responses for the 10 evaluable T/NK-NHL pts (ORR/CR 80%/40%) are shown in Table 1 [ENKTL (n=5; 1 CR 3 PR); T cell (n=5; 3 CR 1 PR)]. Two pts (ENKTL and PTCL-NOS) in PR/CR respectively were withdrawn for autologous stem cell transplantation (ASCT). For DLBCL (n=6), ORR/CR was 66%/33% (both CRs were in pts refractory to first-line R-CHOP). For IA-LPD (predominantly B-cell), ORR/CR was 30%/20% (PTLD: 1 CR, other: 1 CR, 1 PR). The median DoR for all responders is 10.6 months, with a median follow-up from response of 5.0 m (range 0.4-22.9 m).

Conclusions: Co-administration of oral Nstat with VGCV has a favorable safety profile, may be suitable for combination with additional agents, and shows promising efficacy in pts with a variety of R/R, heavily pre-treated EBV+ lymphomas, the majority of whom were lacking therapeutic options. Preliminary data suggests that this regimen is highly active in EBV+T/NKNHL pts who are refractory to standard therapies, including ASCT (Table 9), and promising in EBV+ DLBCL. Thus far, no apparent correlation was noted between degree of EBER positivity in pre-study tumor biopsies and ORR.

TABLE 9

| | | | Refractory | | |
|---|---|---|---|---|---|
| Age, | | Prior lines | to last | | Response |
| Gender | Subtype | of therapy | therapy | Response | duration |
| 60, F | ENKTL | IMEP, GDP, | Y | CR | 3.7 m |
| | | nivolumab + | | | |
| | | EBV-specific | | | |
| | | T cells | | | |
| 44, M | ENKTL | SMILE, | Y | PR | 2.7 m |
| | | ASCT, | | | |
| | | pembrolizumab | | | |
| 48, M | ENKTL | SMILE, | N | PR | 3.5 m |
| | | SMILE, | | | (w/d for |
| | | cisplatin + | | | ASCT) |
| | | radiotherapy | | | |
| 37, F | ENKTL | SMILE + | Y | PR | 9.4 m |
| | | XRT, ASCT | | | (ongoing) |
| 47, F | ENKTL | SMILE | Y | PD | — |
| 58, M | PCTL- | CHOEP, | Y | PR | 10.6 m |
| | NOS | romidepsin, | | | |
| | | ASCT, | | | |
| | | romidepsin | | | |
| 74, F | PCTL- | CHOP | Y | CR | 5.8 m |
| | NOS | | | | (w/d for |
| | | | | | ASCT) |
| 63, M | AITL | CHOP, ICE | Y | CR | 10.8 m |
| 43, M | CTCL | allosq, A- | Y | PD | — |
| | | dmDT390- | | | |
| | | bisFv (anti-T- | | | |
| | | cell | | | |
| | | immunotoxin), | | | |
| | | bexarotene, | | | |
| | | vorinostat | | | |
| 66, M | AITL | CHOP | Y | CR | 0.4 m |
| | | | | | (ongoing) |

S: steroid (dexamethasone),
M: methotrexate,
I: ifosfamide,
L: L-asparaginase,
E: etoposide
C: cyclophosphamide
H: doxorubicin
0: vincristine
P: prednisone (P: cisplatin in GDP)
D: dexamethasone
w/d withdrawn,
XRT: radiation therapy

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

What is claimed is:

1. A method of treating a cancer in an individual, the method comprising administering to the individual: (a) an effective amount of a histone deacetylase inhibitor (HDACi); and (b) an effective amount of an antiviral drug, wherein the effective amount of the HDACi is about 75% or less of a maximum tolerated dose.

2. The method of claim 1, wherein the effective amount of the HDACi is about 50% or less of the maximum tolerated dose.

3. The method of claim 1, wherein the effective amount of the HDACi is about 30% or less of the maximum tolerated dose.

4. The method of claim 1, wherein the effective amount of the HDACi is about 25% or less of the maximum tolerated dose.

5. The method of claim 1, wherein the effective amount of the HDACi is about 20% or less of the maximum tolerated dose.

6. The method of claim 1, wherein the effective amount of the HDACi is about 15% or less of the maximum tolerated dose.

7. The method of claim 1, wherein the effective amount of the HDACi is about 10% or less of the maximum tolerated dose.

8. The method of any one of claims 1 to 7, wherein the effective amount of a histone deacetylase inhibitor is an amount with a low probability of a grade 3 or 4 adverse event.

9. The method of claim 8, wherein the low probability of a grade 3 or 4 adverse event is less than about 10%.

10. The method of claim 8, wherein the low probability of a grade 3 or 4 adverse event is less than about 5%.

11. The method of any one of claims 1 to 10, wherein the HDACi is administered orally.

12. The method of any one of claims 1 to 11, wherein the HDACi is any one or more selected from the list consisting of: vorinostat, romidepsin, mocetinostat, belinostat, pracinostat, givinostat, panobinostat, CUDC-101, CDX101, chidamide, and nanatinostat.

13. The method of any one of claims 1 to 12, wherein cytotoxic activity of the antiviral agent is activated by a viral kinase.

14. The method of any one of claims 1 to 13, wherein the viral kinase is a human herpes virus thymidine kinase, an Epstein-Barr virus thymidine kinase, an Epstein-Barr virus protein kinase, or a CMV protein kinase.

15. The method of any one of claims 1 to 14, wherein the antiviral agent is selected from the list consisting of aciclovir, ganciclovir, valaciclovir, valganciclovir, zidovudine, and famciclovir.

16. The method of any one of claims 1 to 15, wherein the antiviral agent is valganciclovir.

17. The method of any one of claims 1 to 16, wherein the antiviral agent is administered at a total daily dose of 1,800 milligrams.

18. The method of any one of claims 1 to 16, wherein the antiviral agent is administered at a total daily dose of 900 milligrams.

19. The method of any one of claims 1 to 16, wherein the antiviral agent is administered at a total daily dose of 450 milligrams.

20. The method of any one of claims 1 to 19, wherein the cancer is a solid tissue cancer.

21. The method of claim 20, wherein the solid tissue cancer is salivary gland cancer, nasopharyngeal carcinoma, head and neck cancer, gastric cancer, a colorectal cancer, breast cancer, glioblastoma, prostate cancer, renal cancer, leiomyosarcoma, pancreatic cancer, or lung cancer.

22. The method of claim 21, wherein the solid tissue cancer is salivary gland cancer, nasopharyngeal carcinoma, head and neck cancer, gastric cancer, a colorectal cancer, or leiomyosarcoma.

23. The method of any one of claims 1 to 19, wherein the cancer is a leukemia or a lymphoma.

24. The method of claim 23, wherein the leukemia or lymphoma is a B cell leukemia or lymphoma.

25. The method of claim 23, wherein the leukemia or lymphoma is a T cell leukemia or lymphoma.

26. The method of claim 23, wherein the leukemia or lymphoma is non-Hodgkin's lymphoma.

27. The method of claim 23, wherein the leukemia or lymphoma is Epstein-Barr Positive T or NK cell non-Hodgkin's lymphoma.

28. The method of claim 23, wherein the leukemia or lymphoma is Hodgkin's lymphoma.

29. The method of any one of claims 1 to 19, wherein the cancer is a cytomegalovirus virus positive cancer.

30. The method of any one of claims 1 to 19, wherein the cancer is an Epstein-Barr virus positive cancer.

31. The method of any one of claims 1 to 30, wherein the individual is treated according to a treatment schedule, wherein the individual is not administered the HDACi for at least one day of the treatment schedule.

32. The method of claim 31, wherein the individual is not administered the HDACi for at least two days of the treatment schedule.

33. The method of claim 31, wherein the individual is not administered the HDACi for at least three days of the treatment schedule.

34. The method of claim 31, wherein the individual is not administered the HDACi for at least four days of the treatment schedule.

35. The method of claim 31, wherein the individual is not administered the HDACi for at least five days of the treatment schedule.

36. The method of claim 31, wherein the treatment schedule has a duration of one week.

37. The method of any one of claims 1 to 36, wherein the HDACi is characterized by an elimination half-life of less than about 30 hours.

38. The method of any one of claims 1 to 36, wherein the HDACi is characterized by an elimination half-life of less than about 15 hours.

39. The method of any one of claims 1 to 36, wherein the HDACi is characterized by an elimination half-life of less than about 12 hours.

40. The method of any one of claims 1 to 36, wherein the HDACi is characterized by an elimination half-life of less than about 8 hours.

41. The method of any one of claims 1 to 36, wherein the HDACi is characterized by an elimination half-life of less than about 4 hours.

42. The method of any one of claims 1 to 36, wherein the HDACi is characterized by an elimination half-life of less than about 2 hours.
